(19) United States
(12) Patent Application Publication
(10) Pub. No.: US 2025/0381194 A1
(43) Pub. Date: Dec. 18, 2025

(54) METHODS FOR MITIGATING THE EFFECTS OF LOW-LEVEL BLASTS THROUGH PHOSPHODIESTERASE-5 INHIBITION

(71) Applicants: University of Kentucky Research Foundation, Lexington, KY (US); THE U.S. GOVERNMENT AS REPRESENTED BY THE DEPT OF VETERANS AFFAIRS, Washington, DC (US)

(72) Inventor: William Brad Hubbard, Nicholasville, KY (US)

(21) Appl. No.: 19/239,527

(22) Filed: Jun. 16, 2025

Related U.S. Application Data
(60) Provisional application No. 63/660,330, filed on Jun. 14, 2024.

(51) Int. Cl. A61K 31/519; A61P 25/00

(52) U.S. Cl. CPC A61K 31/519; A61P 25/00

(57) ABSTRACT

A method for treating low-level blast traumatic brain injury includes administering a phosphodiesterase-5 (PDE5) inhibitor, such as sildenafil, to a subject following exposure of the subject to one or more low-level blasts. Administration of the PDE5 inhibitor may prove useful with respect to improving the vascular integrity of a subject by increasing brain capillary respiration, mitochondrial respiration, mitochondrial density, mitochondrial biogenesis, astrocyte level or homeostasis, and/or tight junction protein expression in the brain of the subject and/or inducing positive metabolic change in the subject by increasing NAD⁺/NADH metabolism. Methods for mitigating the effects of blast injury on the vascular integrity of a subject and inducing metabolic change are also provided.

### CROSS-REFERENCE TO RELATED APPLICATIONS

The present application claims priority to U.S. Patent Application Ser. No. 63/660,330 filed on Jun. 14, 2024, the entire disclosure of which is incorporated herein by reference.

### GOVERNMENT INTEREST

This invention was made with government support under grant number IK2 BX004618, awarded by the United States Department of Veteran Affairs. The government has certain rights in the invention.

### TECHNICAL FIELD

The presently disclosed subject matter generally relates to methods for mitigating the adverse effects of low-level blast exposure in a subject. In particular, certain embodiments of the presently disclosed subject matter relate to methods for mitigating the effects of low-level blast exposure in a subject through phosphodiesterase-5 (PDE5) inhibition.

### BACKGROUND

Long-term consequences of military-related and deployment-related occupational exposures can continue to affect Veterans throughout their lives. Among these are occupational exposure to low-level blasts (LLBs), which can also be characterized as “mild blasts,” that soldiers routinely are exposed to during training operations, breaching activity, and tour of duty. Currently, there is much concern about traumatic brain injury (TBI) from blast exposure, especially in conjunction with post-traumatic stress disorder (PTSD) symptoms. It is well known that Veterans suffer from PTSD, whether related or unrelated to sustaining TBI. However, TBI and PTSD are often associated together in the literature and are proposed to have a direct relationship. Even more concerning is the evidence that mild blast exposure, with any diagnosed concussion, can lead to long-term neuropsychological deficits. Furthermore, military personnel that sustain mild blasts without any loss of consciousness may be more pre-disposed to develop PTSD, as PTSD occurs less frequently when there is memory loss of the traumatic event. The symptomology associated with mild blast exposure can be quite insidious, without overt acute damage but resulting in chronic emotional and behavioral deficits. Mild blasts can be categorized as an occupational exposure but, over time, turns into a neurological injury.

### SUMMARY

The presently disclosed subject matter meets some or all of the above-identified limitations, as will become evident to those of ordinary skill in the art after a study of information provided in this document.

This summary describes several embodiments of the presently disclosed subject matter, and in many cases lists variations and permutations of these embodiments and implementations. This summary is merely exemplary of the numerous and varied embodiments and implementations. Mention of one or more representative features of a given embodiment or implementation is likewise exemplary. Such an embodiment or implementation can typically exist with or without the feature(s) mentioned; likewise, those features can be applied to other embodiments or implementations of the presently disclosed subject matter, whether listed in this summary or not. To avoid excessive repetition, this summary does not list or suggest all possible combinations of such features.

The present disclosure includes methods for treating low-level blast traumatic brain injury (TBI) in a subject.

A method for treating low-level blast TBI in accordance with embodiments of the present disclosure, includes administering a phosphodiesterase-5 (PDE5) inhibitor to a subject following exposure to one or more low-level blasts. In some embodiments, the one or more low-level blasts have a force that is less than 17 pounds per square inch (PSI). In some embodiments, the one or more low-level blasts have a force that is between 4 PSI and 17 PSI.

In some embodiments of the method for treating low-level blast TBI, the PDE5 inhibitor is sildenafil.

In some embodiments of the method for treating low-level blast TBI, the PDE5 inhibitor is administered to the subject within 24 hours following exposure of the subject to a low-level blast of the one or more low-level blasts. In some embodiments, the PDE5 inhibitor is administered to the subject within one hour following exposure of the subject to a low-level blast of the one or more low-level blasts. In some embodiments, administering the PDE5 inhibitor comprises multiple administrations of the PDE5 inhibitor to the subject. In some embodiments, administering the PDE5 inhibitor comprises administering the PDE5 inhibitor to the subject daily for a period of at least seven days.

In some embodiments of the method for treating low-level blast TBI, the subject is exposed to multiple low-level blasts. In some embodiments, a first administration of the PDE5 inhibitor is administered to the subject following exposure of the subject to a first low-level blast, and a second administration of the PDE5 inhibitor is administered to the subject following exposure of the subject to a second low-level blast occurring after the first administration of the PDE5 inhibitor. In some embodiments, the first administration of the PDE5 inhibitor is administered within following 24 hours of exposure to the first-occurring low-level blast of multiple low-level blasts to which the subject is exposed. In some embodiments, the PDE5 inhibitor is first administered to the subject following exposure of the subject to the last-occurring low-level blast of the multiple low-level blasts to which the subject is exposed. In some embodiments, the subject is without chronic traumatic brain injury prior to exposure to the one or more low-level blasts.

The present disclosure further includes methods for mitigating the effects of blast injury on the vascular integrity of a subject.

An exemplary method for mitigating the effects of blast injury on the vascular integrity of a subject in accordance with embodiments of the present disclosure includes administering PDE5 inhibitor to the subject following exposure of the subject to one or more blasts to thereby increase at least one of brain capillary respiration, mitochondrial respiration, mitochondrial density, mitochondrial biogenesis, astrocyte levels or homeostasis, and tight junction protein expression in a brain of the subject.

In some embodiments of the method for mitigating the effects of blast injury on the vascular integrity of a subject, the PDE5 inhibitor is sildenafil.

In some embodiments of the method for mitigating the effects of blast injury on the vascular integrity of a subject, the PDE5 inhibitor is administered to the subject within 24 hours following exposure of the subject to a low-level blast of the one or more low-level blasts.

In some embodiments of the method for mitigating the effects of blast injury on the vascular integrity of a subject, administration of the PDE5 inhibitor increases at least one of cyclic guanosine monophosphate (cGMP), proliferator-activated receptor gamma coactivator 1-alpha (PGC-1α), glial fibrillary acidic protein (GFAP), translocase of the outer mitochondrial membrane complex subunit 20 (TOM20), and zonula occludens-1 (ZO-1) in the subject. In some embodiments of the method for mitigating the effects of blast injury on the vascular integrity of a subject, the one or more blasts comprises multiple low-level blasts. In some embodiments, the PDE5 inhibitor is first administered to the subject within 24 hours following exposure to a first-occurring blast of multiple low-level blasts to which the subject is exposed. In some embodiments, a first administration of the PDE5 inhibitor is administered to the subject following exposure of the subject to a first low-level blast, and a second administration of the PDE5 inhibitor is administered to the subject following exposure of the subject to a second low-level blast occurring after the first administration of the PDE5 inhibitor. In some embodiments, the PDE5 inhibitor is first administered to the subject following exposure to the last-occurring blast of the multiple low-level blasts to which the subject is exposed. In some embodiments, administering the PDE5 inhibitor comprises administering the PDE5 inhibitor daily to the subject for a period of at least seven days.

The present disclosure also includes methods for inducing a metabolic change in a subject.

An exemplary method for inducing a metabolic change in a subject in accordance with embodiments of the present disclosure includes administering an effective amount of a phosphodiesterase-5 (PDE5) inhibitor to a subject following exposure of the subject to one or more low-level blasts to thereby increase NAD⁺/NADH metabolism in the subject. In some embodiments, administration of the PDE5 inhibitor reduces nicotinamide in the subject. In some embodiments, the PDE5 inhibitor is sildenafil.

### BRIEF DESCRIPTION OF THE DRAWINGS

A better understanding of the features and advantages of the present invention will be obtained by reference to the following detailed description that sets forth illustrative embodiments, in which the principles of the invention are used, and the accompanying drawings of which:

FIG. 1A is a graph showing representative pressure-time traces disclosed in Reneer, et al. (2011) utilizing a McMillan blast device (MBD) (RDX, cyclotrimethylenetrinitramine) and a free-field (side-on) sensor.

FIG. 1B is a graph showing a representative curve of static overpressure/underpressure with respect to time measured by a side-on piezoresistive sensor positioned coincident with the animal holder.

FIG. 1C is a graph showing an overlay of overpressure waves with respect to time. The curve with higher pressure (peak of 24 PSI) was generated using a face-on sensor of a pilot static probe, which measures total overpressure. The lower curve (peak of 11 PSI) was generated using a side-on sensor of a pitot statistic probe, which measures static overpressure. Shaded areas represent dynamic overpressure impulses as a relatively minor component of the blast wave.

FIGS. 2A-2I. Mild blast TBI (mbTBI) alters exploratory and anxiety-related behavior in male rats but not in female rats. Rats were subjected to either sham or single mbTBI procedures. Open field test (FIGS. 2A-2E) and elevated plus maze (FIGS. 2F-2I) from male and female rats. FIG. 2A is a bar graph representing the total duration (seconds) of time to a center area of an open field during acclimatization (n=11-12/group). FIG. 2B is a bar graph representing the frequency of entrances to a center area of open field during acclimatization (n=11-12/group). FIG. 2C is a bar graph representing the total duration (seconds) of time in a center area of open field at two days post-mbTBI (n=11-12/group). FIG. 2D is a bar graph representing the frequency of entrances to center area of open field at two days post-mbTBI (n=11-12/group). FIG. 2E are representative heat maps from open field test at two days post mbTBI from male and female groups. FIG. 2F is a representative heat map from an elevated plus maze for a male mbTBI animal. FIG. 2G is a graph showing the latency to enter open arm (seconds) in elevated plus maze at seven days post-mbTBI (n=6/group). FIG. 2H is a graph showing the total time spent in open arm (seconds) in an elevated plus maze at seven days post-mbTBI (n=6/group). FIG. 2I is a graph showing the number of open arm entrances in an elevated plus maze at seven days post-mbTBI (n=6/group). Data represented as Mean±SEM. P≤0.05*, P≤0.01** by Two-way ANOVA with Bonferroni post-hoc.

FIG. 3 is a graph showing recognition index (RI) at seven days after repeated mild-blast traumatic brain injury (rmbTBI). Rats were subjected to either sham or repeated (2 blasts separated by 24 h) (RI=Time spent with novel object/time spent with both objects). Sham animals explored the novel object (as an index of proper cognitive function) significantly more than chance. rmbTBI displayed a lack of learning or cognitive function. Data represented as Mean±SEM. P≤0.001*** using one-sample t-test.

FIGS. 4A-4E. mbTBI affects blood-brain barrier integrity in male rats but not in female rats. SMI-71 immunofluorescence to assess capillary barrier integrity. FIG. 4A is a series of representative SMI-71 immunofluorescence micrograph images from sham and mbTBI rat brain at 24 h post-mbTBI (top), with 20× magnification of vascular integrity in amygdala region (bottom). FIG. 4B is a graph showing vascular density quantification using ImageJ in the whole brain section of sham and mbTBI from male and female rats 24 h post-mTBI (n=3-6/group). FIG. 4C is a graph showing vascular density quantification using ImageJ in the amygdala region of sham and mbTBI from male and female rats 24 h post-mTBI (n=3-6/group). FIG. 4D is a graph showing vascular density quantification using ImageJ in whole brain section of sham and mbTBI from male and female rats seven days post-mbTBI (n=3-4/group). FIG. 4E is a graph showing vascular density quantification using ImageJ in the amygdala region of sham and mbTBI from male and female rats seven days post-mbTBI (n=3-4/group). Data represented as Mean±SEM. P≤0.05* by Two-way ANOVA with Bonferroni post-hoc.

FIGS. 5A-5C. rmbTBI decreases astrocyte endfeet (AQP4) expression in male but not female rats. rmbTBI groups that received 2 blasts separated by 1 h (rmb-TBI-1 hr) or 2 blasts separated by 24 h (rmbTBI-24 hr) were compared. FIG. 5A is a series of images showing representative AQP4 immunofluorescence of brain and high mag (20×) (bottom) of cortex from male sham (left) and repeated mild-blast traumatic brain injury (rmbTBI) groups (middle=rmbTBI-1 hr, right=rmbTBI-24 hr) at seven days post-rmbTBI. FIG. 5B is a graph showing AQP4 fluorescence quantification using HALO software of sham and rmbTBI groups from male rats seven days post-rmbTBI (n=5-6/group). FIG. 5C is a graph showing AQP4 fluorescence quantification using HALO software of sham and rmbTBI groups from female rats seven days post-rmbTBI (n=5-6/group). Data represented as Mean±SEM. P≤0.01**; P≤0.001*** by one-way ANOVA with Tukey post-hoc.

FIGS. 6A-6D. mbTBI decreases astrocyte coverage around brain capillaries. GFAP and SMI-71 double immunostaining to assess astrocyte coverage around the brain capillary. FIG. 6A is a series of confocal micrograph images (GFAP-green; SM-71-red; Hoechst-blue) GFAP around SMI-71+ brain capillary from the amygdala (sham and mbTBI; n=3 rats/group) of male and female rats. FIG. 6B is a graph showing the quantification of GFAP around SMI-71+ brain capillary from amygdala (sham and mbTBI; n=3 rats/group) of male rats (25 vessels from each rat brain amygdala region; n=75 vessels/group). FIG. 6C is a graph showing the quantification of GFAP around SMI-71+ brain capillary from amygdala (sham and mbTBI; n=3 rats/group) of female rats (25 vessels from each rat brain amygdala region; n=75 vessels/group).

FIG. 6D is a series of high-resolution micrographs (GFAP-green; SMI-71-red; Hoechst-blue) of brain vessel and surrounding astrocytes from male sham and mbTBI rats. Data represented as Mean±SEM. P≤0.0001**** two-tailed, unpaired t test.

FIG. 7 is a series of graphs showing Western blot densitometry quantification of PDGFR-β, GFAP, and IBA-1 proteins from the cortex region of male rats at seven days following rmbTBI (2 blasts separated by 24 h interval) (n=6/group). Data represented as Mean±SEM. P<0.05* by unpaired two-tailed t-test. mbTBI decreases pericyte and astrocyte markers while increasing microglial marker in cortex of male rats.

FIGS. 8A-8C. rmbTBI alters tight junction proteins in male rats but not in female rats. Tight junction (TJ) proteins, occludin, ZO-1, and claudin 5, were stained in isolated cortical capillaries using immunofluorescence. FIG. 8A is a series of representative immunofluorescence micrograph from male sham and rmbTBI groups at seven days post-rmbTBI. FIG. 8B is a series of graphs showing tight junction (TJ proteins) (occluding, zonula occludens-1 (ZO-1), claudin-5) fluorescence quantification using Nikon software of sham and rmbTBI groups from male rats seven days post-rmbTBI (n=25-30 capillaries from n=6 rats/group). FIG. 8C is a series of graphs showing TJ proteins (ZO-1, occluding) fluorescence quantification using Nikon software of sham and rmbTBI groups from female rats seven days post-rmbTBI (n=25-30 capillaries from n=6 rats/group). Blast occurred at 1 h or 24 h intervals. Data represented as Mean±SEM. P≤0.0001**** by one-way ANOVA with Tukey post-hoc.

FIG. 9 is a series of graphs showing basal respiration, proton leak, maximal respiration, adenosine triphosphate (ATP), and basal glycolysis of capillaries isolated from the cortex of a first cohort of female and male Sprague Dawley rats. Isolated capillaries from the first cohort were divided into (n=4/group): control sham; sildenafil sham (capillaries incubated with sildenafil); control blast (single blast exposure at 11 PSI); and sildenafil blast (single blast exposure at 11 PSI and incubated with sildenafil for two hours at one day after blast exposure).

FIG. 10 is a series of graphs showing basal respiration, maximal respiration, and basal glycolysis of capillaries isolated from the cortex of a second cohort of female and male Sprague Dawley rats. Isolated capillaries from the second cohort were divided into (n=4/sez/group): control sham; sildenafil sham (capillaries incubated with sildenafil); control blast (single blast exposure at 11 PSI); and sildenafil blast (single blast exposure at 11 PSI and incubated with sildenafil for two hours at one day after blast exposure).

FIG. 11 is a graph showing the combined maximal respiration of respective groups from first cohort (FIG. 9) and second cohort (FIG. 10) of capillaries isolated from the cortex of Sprague Dawley rats.

FIGS. 12A-12B. Mitochondrial footprint is increased after sildenafil compared to vehicle in cell culture. FIG. 12A is a series of images showing rat brain microvascular endothelial cells incubated with sildenafil (1 μM in 1% DMSO diluted in cell culture media) or vehicle (1% DMSO diluted in cell culture media) overnight and labeled with mitotracker green to examine mitochondrial biogenesis. FIG. 12B is a series of graphs showing the percentage of mitochondrial signal footprint, mitochondrial branching length (μm), mitochondrial network branching mean (μm), and summed mitochondrial branch length (μm) fibroblast incubated with sildenafil versus vehicle. Mitochondrial Network Analysis (MiNA) workflow in ImageJ was used to examine mitochondrial parameters. Sildenafil administration increased mitochondrial footprint/density, based on cell area, compared to vehicle. Mitochondrial branching is decreased after sildenafil compared to vehicle, which could be due biogenesis-mediated mitochondrial fission processes.

FIG. 13 is a schematic diagram of a model for sildenafil treatment for low-level blast TBI and impact on mitochondrial restoration.

FIG. 14 is a graph showing cyclic guanosine monophosphate (cGMP) in isolated control capillaries and isolated sildenafil-treated capillaries (100 nM, 1 μM, or 10 μM) obtained from rat cortex. Naïve brain capillaries (not subjected to mbTBI or rmbTBI exposure) were isolated from rat cortex and incubated for two hours and then media was collected and tested using cGMP ELISA kit.

FIGS. 15A-15B. Sildenafil treatment produces increases in mitochondrial biogenesis and astrocyte homeostasis in cortex tissue at seven days post-rmbTBI. FIG. 15A is an image showing Western blots for PGC-1α, GFAP, and β-actin in sham subjects administered daily with vehicle versus rmbTBI (2 blasts separated by 24 h interval) subjects administered daily with vehicle or sildenafil treatment (5 mg/kg low dose or 20 mg/kg high dose) immediately following sham or immediately following each blast exposure during rmbTBI procedure and continuing daily to day seven. FIG. 15B is a series of graphs showing quantification of PGC-1α and GFAP levels between groups (n=5 rats/group). Data represented as Mean±SEM. Cortical tissue was extracted at seven days post-rmbTBI and probed for PGC-1α and GFAP levels.

FIGS. 16A-16B. Sildenafil treatment increased mitochondrial biogenesis in hippocampal tissue seven days post-rmbTBI. FIG. 16A is an image showing Western blots for PGC-1a in sham subjects administered daily with vehicle versus rmbTBI (2 blasts separated by 24 h interval) subjects administered daily with vehicle or sildenafil treatment (5 mg/kg low dose or 20 mg/kg high dose) immediately following sham or immediately following each blast exposure during rmbTBI procedure and continuing daily to day seven. FIG. 16B is a graph showing quantification of PGC-1α levels between groups. Sham and rmbTBI Sild High groups are significantly increased compared to rmbTBI vehicle (n=5 rats/group). Data represented as Mean±SEM. P≤0.001***P≤0.05* by one-way ANOVA with Tukey's post-hoc.

FIGS. 17A-17B. PDE5 inhibition increased capillary-specific mitochondrial content at seven days post-rmbTBI. FIG. 17A is a series of representative images showing stained capillaries treated with vehicle or sildenafil low-dose sildenafil (5 mg/kg low dose or 20 mg/kg high dose) immediately following each blast exposure during rmbTBI (2 blasts separated by 24 h interval) procedure and continuing daily to day seven. Capillaries were isolated at seven days post-rmbTBI and stained for mitochondria marker translocase of outer mitochondrial membrane 20 (TOM20) (red), isolectin (green), and DAPI (blue). FIG. 17B is a graph showing quantification of mitochondrial volume between capillaries treated with vehicle or sildenafil low-dose sildenafil (5 mg/kg low dose or 20 mg/kg high dose) immediately following each blast during rmbTBI procedure and continuing daily to day seven. Sild High groups have increased volume compared to rmbTBI vehicle (n=3 rodents/group). Data represented as Mean±SEM. P≤ 0.001*** by one-way ANOVA with Dunnett's post-hoc.

FIGS. 18A-18B. Sildenafil treatment non-significantly increased tight junction integrity at seven days post-rmbTBI. FIG. 18A is a series of images showing stained cortical capillaries of sham and rmbTBI (2 blasts separated by 24 h interval) subjects treated with vehicle or sildenafil low-dose sildenafil (5 mg/kg low dose or 20 mg/kg high dose) immediately following each blast during rmbTBI procedure and continuing daily to day seven. Cortical capillaries were isolated at seven days post-rmbTBI and immunostained for ZO-1. FIG. 18B is a graph showing quantification of ZO-1 fluorescence levels between groups sham and rmbTBI subjects treated with vehicle or sildenafil low-dose sildenafil (5 mg/kg low dose or 20 mg/kg high dose) immediately following each blast exposure during rmbTBI procedure and continuing daily to day seven. rmbTBI Sild High groups are increased compared to rmbTBI vehicle (n=15 capillaries from 5 rodents/group). Data represented as Mean±SEM. P≤0.05* by one-way ANOVA with Tukey's post-hoc.

FIG. 19 is a graph illustrating sildenafil treatment improves cognitive performance at three days post-rmbTBI (2 blasts separated by 24 h interval). Animals were administered daily vehicle or sildenafil treatment (5 mg/kg low dose or 20 mg/kg high dose) immediately following each blast exposure. Sham and rmbTBI Sild Low groups have significantly increased recognition index (RI) as compared to chance (0.5) (n=4-5 rodents/group). Data represented as Mean±SEM. P≤0.05* by one-sample t-test (see Carlson, et al. (2018) as reference for statistical analysis in TBI research).

FIG. 20 is a graph showing the Cortex Metabolomic Profile of rmbTBI+Vehicle versus rmbTBI+Sildenafil. The volcano plot showing differential metabolite expression at seven days following injury in the cortex of rmbTBI rats (2 blasts separated by 24 h interval) treated with daily vehicle versus 20 mg/kg sildenafil (administration following each respective blast exposure and continuing daily to day seven). Each point represents a metabolite; the x-axis indicates log 2 fold change (FC), and the y-axis indicates-log₁₀ (p-value). Statistical significance was determined using an unpaired t-test (p=0.05).

FIG. 21A is a graph showing the relative abundance of NADH in the cortex of sham versus rmbTBI subjects (experimental design as in FIG. 20) treated with daily vehicle or 20 mg/kg sildenafil (administration following each respective blast exposure and continuing daily to day seven).

FIG. 21B is a graph showing the relative abundance of NAD in the cortex of sham versus rmbTBI subjects (experimental design as in FIG. 20) treated with daily vehicle or 20 mg/kg sildenafil (administration following each respective blast exposure and continuing daily to day seven).

FIG. 21C is a graph comparing the relative abundance of nicotinamide in the cortex of sham versus rmbTBI subjects (experimental design as in FIG. 20) treated with daily vehicle or 20 mg/kg sildenafil (administration following each respective blast exposure and continuing daily to day seven). The asterisk (*) indicates a statistically significant reduction in nicotinamide abundance in the rmbTBI+sildenafil group compared to rmbTBI+vehicle. The comparisons between groups were statistically analyzed using a one-way ANOVA.

FIG. 22A is a graph showing the relative abundance of cyclic adenosine monophosphate (cAMP) in the cortex of sham versus rmbTBI subjects (experimental design as in FIG. 20) treated with daily vehicle or 20 mg/kg sildenafil (administration following each respective blast exposure and continuing daily to day seven).

FIG. 22B is a graph comparing the relative abundance of guanosine-5-monophosphate (GMP) in the cortex of sham versus rmbTBI subjects (experimental design as in FIG. 20) treated with daily vehicle or 20 mg/kg sildenafil (administration following each respective blast exposure and continuing daily to day seven). A significant increase (p<0.01) in GMP levels is observed in the rmbTBI+vehicle group compared to Sham.

FIG. 23A is a graph depicting the relative abundance of fructose-6-phosphate. in the cortex of sham versus rmbTBI subjects (experimental design as in FIG. 20) treated with daily vehicle or 20 mg/kg sildenafil (administration following each respective blast exposure and continuing daily to day seven). A significant increase (p<0.05) is observed in the rmbTBI+vehicle group compared to Sham.

FIG. 23B is a graph comparing the relative abundance of glucose-6-phosphate in the cortex of sham versus rmbTBI subjects (experimental design as in FIG. 20) treated with daily vehicle or 20 mg/kg sildenafil (administration following each respective blast exposure and continuing daily to day seven). A significant increase (p<0.05) is observed in the rmbTBI+vehicle group compared to Sham.

FIG. 24 is a diagram showing a timeline for a chronic, delayed sildenafil study to examine chronic effects of rmbTBI and efficacy of sildenafil treatment. Data shown in FIGS. 25A-33 generated following experimental design reflected in FIG. 24.

FIG. 25A is a series of images showing ionized calcium-binding adapter molecule 1 (IBA1) expression in the cortex and corpus collosum of sham, rmbTBI, and rmbTBI sildenafil-treated subjects five months post-blast treatment.

FIG. 25B is a series of graphs showing IBA1 expression in the cortex, hippocampus, amygdala, and corpus collosum of sham, rmbTBI, and rmbTBI sildenafil-treated subjects five months post-blast treatment. Percent Alexa Fluor 594 Area±SEM shown for the cortex (p=0.3500), hippocampus (p=0.5765), amygdala (p=0.9434), and corpus callosum (p=0.1002) analyzed by one-way ANOVA (n=11-12/group).

FIG. 26A is a graph showing cortex glial fibrillary acidic protein (GFAP) expression in blood vessels versus other tissue following rmbTBI and sildenafil treatment at five months post-blast treatment. EGFP average positive intensity±SEM, analyzed by two-way ANOVA (*=p<0.05, **=p<0.01, ***=p<0.001, ****=p<0.0001). Halo AI was trained to recognize blood vessels on 40 micron brain slices (n=11-12/group).

FIG. 26B is a graph showing hippocampus GFAP expression in blood vessels versus other tissue following rmbTBI and sildenafil treatment at five months post-blast treatment. EGFP average positive intensity±SEM, analyzed by two-way ANOVA (*=p<0.05, **=p<0.01, p<0.001, ****=p<0.0001). Halo AI was trained to recognize blood vessels on 40 micron brain slices (n=11-12/group).

FIG. 26C is a graph showing amygdala GFAP expression in blood vessels versus other tissue following rmbTBI and sildenafil treatment at five months post-blast treatment. EGFP average positive intensity±SEM, analyzed by two-way ANOVA (*=p<0.05, **=p<0.01, p<0.001, ****=p<0.0001). Halo AI was trained to recognize blood vessels on 40 micron brain slices (n=11-12/group).

FIG. 26D is a graph showing corpus callosum GFAP expression in blood vessels versus other tissue following rmbTBI and sildenafil treatment at five months post-blast treatment. EGFP average positive intensity±SEM, analyzed by two-way ANOVA (*=p<0.05, ***=p<0.01, ***=p<0.001, ****=p<0.0001). Halo AI was trained to recognize blood vessels on 40 micron brain slices (n=11-12/group).

FIG. 27A is a graph showing cortex IBA1 expression in blood vessels versus other tissue following rmbTBI and sildenafil treatment at five months post-blast treatment. Alexa Fluor 594 average positive intensity±SEM, analyzed by two-way ANOVA (*=p<0.05, **=p<0.01, ***=p<0.001, ****=p<0.0001). Halo AI was trained to recognize blood vessels on 40 micron brain slices (n=11-12/group).

FIG. 27B is a graph showing hippocampus IBA1 expression in blood vessels versus other tissue following rmbTBI and sildenafil treatment at five months post-blast treatment. Alexa Fluor 594 average positive intensity±SEM, analyzed by two-way ANOVA (*=p<0.05, **=p<0.01, ***=p<0.001, ****=p<0.0001). Halo AI was trained to recognize blood vessels on 40 micron brain slices (n=11-12/group).

FIG. 27C is a graph showing amygdala IBA1 expression in blood vessels versus other tissue following rmbTBI and sildenafil treatment at five months post-blast treatment. Alexa Fluor 594 average positive intensity±SEM, analyzed by two-way ANOVA (*=p<0.05, **=p<0.01, ***=p<0.001, ****=p<0.0001). Halo AI was trained to recognize blood vessels on 40 micron brain slices (n=11-12/group).

FIG. 27D is a graph showing corpus callosum IBA1 expression in blood vessels versus other tissue following rmbTBI and sildenafil treatment at five months post-blast treatment. Alexa Fluor 594 average positive intensity±SEM, analyzed by two-way ANOVA (*=p<0.05, **=p<0.01, ***=p<0.001, ****=p<0.0001). Halo AI was trained to recognize blood vessels on 40 micron brain slices (n=11-12/group).

FIGS. 28A-28D. Correlation between mitochondrial volume and tight junction proteins. Isolated brain capillaries from sham-vehicle group (n=6, vehicle treatment), sham-sildenafil group (n=6, treated with 20 mg/kg sildenafil), rmb TBI-vehicle group (n=11, repeated blast injury and vehicle treatment), and rmbTBI-sildenafil group (n=12, repeated blast injury and treated with 20 mg/kg sildenafil daily) were stained for mitochondrial marker and tight junction markers using immunofluorescence. Data from normalized mitochondrial volume and ZO-1 fluorescent intensity were performed for correlation analysis and graphed as XY scatter plots for: FIG. 28A Sham+Veh group, FIG. 28B Sham+Sild group, FIG. 28C rmbTBI+Veh group, and FIG. 28D rmbTBI+Sild group. The Pearson r and two-tailed p value are shown on each plot.

FIG. 29 is a series of graphs showing sildenafil treatment strengthens the correlation between mitochondrial volume and tight junction proteins. Isolated brain capillaries from vehicle group (n=17, vehicle treatment), sildenafil group (n=18, treated with 20 mg/kg sildenafil) were stained for mitochondrial marker and tight junction markers using immunofluorescence. Data from normalized mitochondrial volume and Zo-1 fluorescent intensity were performed for correlation analysis and graphed as XY scatter plots for vehicle group (left) and Sildenafil treatment group (right). The Pearson r and two-tailed p value are shown on each plot.

FIG. 30A is an image showing magnetic resonance imaging (MRI) analysis of cerebral blood flow (CBF) in a region of interest (ROI) in the cortex of a sham at two months following sham procedures. ROI manually delineated using FSL software.

FIG. 30B is a graph showing mean CBF in the cortex of sham, rmbTBI vehicle, and rmbTBI sildenafil treated (20 mg/kg daily) (n=11-12/group) at 2 months and 4.5 months post rmbTBI. Regional CBF values were extracted and analyzed using two-way ANOVA with a mixed-effects model, followed by Tukey's multiple comparisons test. Statistical significance was set at p<0.05.

FIG. 31A is an image showing magnetic resonance imaging (MRI) analysis of cerebral blood flow (CBF) in a region of interest (ROI) in the hippocampus of a sham subject at two months following sham procedures. ROI manually delineated using FSL software.

FIG. 31B is a graph showing mean CBF in the hippocampus of sham, rmbTBI vehicle, and rmbTBI sildenafil treated (20 mg/kg daily) (n=11-12/group) at 2 months and 4.5 months post rmbTBI. Regional CBF values were extracted and analyzed using two-way ANOVA with a mixed-effects model, followed by Tukey's multiple comparisons test. Statistical significance was set at p<0.05.

FIG. 32A is an image showing magnetic resonance imaging (MRI) analysis of cerebral blood flow (CBF) in a region of interest (ROI) in the amygdala of a sham subject at two months following sham procedures. ROI manually delineated using FSL software.

FIG. 32B is a graph showing mean CBF in the amygdala of sham, rmbTBI vehicle, and rmbTBI sildenafil treated (20 mg/kg daily) (n=11-12/group) at 2 months and 4.5 months post rmbTBI.mbtbi Regional CBF values were extracted and analyzed using two-way ANOVA with a mixed-effects model, followed by Tukey's multiple comparisons test. Statistical significance was set at p<0.05.

FIG. 33 is a graph showing a fear conditioning analysis of sham, rmbTBI sildenafil treated (20 mg/kg daily), and rmbTBI vehicle subjects. This behavioral paradigm tests conditioned freezing response paired with foot shocks on day 1, followed by behavior extinction paradigm in days 2-4. Sham group was statistically elevated at day 1 compared to rmbTBI Vehicle. *p<0.05. (n=12/group). Further, Sham and rmbTBI Sildenafil groups showed a significant time-related decrease of freezing episodes, consistent with extinction, that were not observed in rmbTBI Vehicle groups. *p<0.05. (n=12/group) Two-way ANOVA with a mixed-effects model followed by Tukey's multiple comparisons test. Mean±SEM.

### DESCRIPTION OF EXEMPLARY EMBODIMENTS

The details of one or more embodiments of the presently disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

While the terms used herein are believed to be well understood by those of ordinary skill in the art, certain definitions are set forth to facilitate explanation of the presently disclosed subject matter.

Unless defined otherwise, all technical and scientific terms used herein have the same meaning as is commonly understood by one of skill in the art to which the invention(s) belong.

All patents, patent applications, published applications and publications, GenBank sequences, databases, websites and other published materials referred to throughout the entire disclosure herein, unless noted otherwise, are incorporated by reference in their entirety.

Where reference is made to a URL or other such identifier or address, it is understood that such identifiers can change and particular information on the internet can come and go, but equivalent information can be found by searching the internet. Reference thereto evidences the availability and public dissemination of such information.

The present application can “comprise” (open ended) or “consist essentially of” the components of the present invention as well as other ingredients or elements described herein. As used herein, “comprising” is open ended and means the elements recited, or their equivalent in structure or function, plus any other element or elements which are not recited. The terms “having” and “including” are also to be construed as open ended unless the context suggests otherwise.

Following long-standing patent law convention, the terms “a”, “an”, and “the” refer to “one or more” when used in this application, including the claims. Thus, for example, reference to “a cell” includes a plurality of such cells, and so forth.

Unless otherwise indicated, all numbers expressing quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, in some embodiments ±0.1%, in some embodiments ±0.01%, and in some embodiments ±0.001% from the specified amount, as such variations are appropriate to perform the disclosed method.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, the terms “treatment” or “treating” of a particular disease or injury in a subject refers to the medical management of the subject with the intent to cure, ameliorate, reduce, or prevent or slow progression of such disease or injury. As will be recognized by one of ordinary skill in the art, the term “cure” does not refer to the ability to completely remove any and all trace of an injury in all cases.

As used herein, “low-level blast traumatic brain injury” refers to traumatic brain injury resulting from a subject's exposure to one or more low-level blasts. As used herein, a “low-level blast” is understood to mean a blast having a force which is less than about 17 pounds per square inch (PSI). In some embodiments of the disclosed methods in which a subject is subjected to one or more low-level blasts, each low-level blast to which the subject is exposed has a force between 4 PSI and 17 PSI or between 4 PSI and 14 PSI. In some embodiments of such methods, each blast is about 11 PSI.

As used herein, a “mild blast” is understood to have the same meaning as, and is used interchangeably with, “low-level blast,” except where otherwise indicated or context precludes.

Low-level blast traumatic brain injury in a subject can, in some embodiments, be identified in a subject by: the exhibition of physical symptoms consistent with traumatic brain injury, such as headache, dizziness, blurred vision, ringing of the ears, fatigue, sensitivity to light or sound, nausea, or vomiting; the exhibition of cognitive symptoms consistent with traumatic brain injury, such as confusion, difficulty concentrating, memory problems, slurred speech, and low reaction times; and/or the exhibition of behavioral or emotional symptoms consistent with traumatic brain injury, such as exhibition of irritability, mood changes, anxiety, and depression. Additionally or alternatively, low-level blast traumatic brain injury in a subject can, in some embodiments, be identified based on an observed: deficit in brain capillary respiration of a subject; deficit in blood-brain barrier (BBB) integrity of a subject; an increase in oxidative stress in the brain vasculature of a subject; decreased mitochondrial respiration in the brain of the subject; a decrease in mitochondrial density in the brain of the subject; a decrease in mitochondria biogenesis in the brain of the subject; a decrease in astrocyte homeostasis in the brain of the subject; a decrease in tight junction protein expression in the brain of the subject, a decrease in NAD⁺/NADH metabolism in the subject; an increase in nicotinamide metabolite in the subject relative to one or more non-injured controls, the subject prior to blast injury, or accepted values or range of values for such biological parameters indicative of brain function observed in subjects without traumatic brain injury.

Low-level blast traumatic brain injury is inclusive of both mild blast traumatic brain injury (mbTBI) and repetitive mild blast traumatic brain injury (rmbTBI). “Mild blast traumatic brain injury (mbTBI)” as used herein refers to traumatic brain injury occurring as a result of a subject's exposure to a single low-level blast. “Repetitive mild blast traumatic brain injury (rmbTBI)” as used herein refers to traumatic brain injury occurring as a result of a subject's exposure to multiple low-level blasts.

As used herein, the terms “subject” or “subject in need thereof” refer to a target of administration, which optionally displays symptoms related to a particular disease, pathological condition, disorder, or the like. The subject of the methods disclosed herein can be a vertebrate, such as a mammal, a fish, a bird, a reptile, or an amphibian. Thus, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered.

As used herein, the term “administering” refers to any method of providing a pharmaceutical preparation to a subject. Such methods are well known to those skilled in the art and include, but are not limited to, oral administration, transdermal administration, administration by inhalation (intranasal), nasal administration, topical administration, intravaginal administration, ophthalmic administration, intraaural administration, intracerebral administration, rectal administration, and parenteral administration, including injectable such as intravenous administration, intra-arterial administration, intramuscular administration, intraperitoneal injection, and subcutaneous administration. Administration can, in various embodiments, be continuous or intermittent. In various aspects, a preparation can be administered therapeutically; that is, administered to treat an existing disease or condition. In further various aspects, a preparation can be administered prophylactically; that is, administered for prevention of a disease or condition. In some embodiments, oral administration is used. In some embodiments, intravenous (IV) administration is used.

As used herein, the term “effective amount” refers to an amount that is sufficient to achieve the desired result or to have an effect on an undesired condition. For example, a “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or multiple days. Guidance can be found in the literature for appropriate dosages for given classes of pharmaceutical products. In further various aspects, a preparation can be administered in a “prophylactically effective amount”; that is, an amount effective for prevention of a disease or condition. Phosphodiesterase-5 (PDE5) inhibitors can be administered in an effective amount in the various methods disclosed herein. In some embodiments of the methods disclosed herein, a PDE5 inhibitor is administered in a dosage of about 5 mg/kg to about 20 mg/kg.

The present disclosure is based, in part, on the discovery that certain PDE5 inhibitors are effective with respect to mitigating adverse effects stemming from low-level blast exposure. In this regard, it has been surprisingly discovered that PDE5 inhibitor administration following a single low-level blast, between and following multiple blasts, and following the last-occurring blast of multiple low-level blasts to which a subject is exposed can improve brain capillary respiration, mitochondrial respiration, mitochondrial density, mitochondrial biogenesis, astrocyte homeostasis, and/or tight junction protein expression in the brain of a subject, and thereby mitigate the effects of low-level blast-induced traumatic brain injury (TBI).

Accordingly, in one aspect, the presently disclosed subject matter includes a method for treating low-level blast TBI, which includes administering an effective amount of a PDE5 inhibitor to a subject following exposure of the subject to one or more low-level blasts.

In some embodiments of the method for treating low-level blast TBI, the PDE5 inhibitor is administered to treat acute low-level blast TBI. Accordingly, in some embodiments, the method includes administering the PDE5 inhibitor to the subject prior to a time period in which any chronic TBI effects or ailments would typically be recognized or expressed in a subject. In various embodiments, administration of the PDE5 inhibitor to the subject can first occur within six months, within five months, within four months, within three months, within two months, within one month, within 14 days, within seven days, within 24 hours, within 1 hour, or within 15 minutes of the subject being exposed to a low-level blast. Thus, in some embodiments, the subject to which treatment is provided does not have (i.e., is without) chronic TBI prior to the subject's exposure to the one or more low-level blasts prompting initiation of treatment.

In some embodiments of the method for treating low-level blast TBI, multiple administrations of PDE5 inhibitor are given to the subject. In some embodiments, the PDE5 inhibitor is administered to the subject daily for multiple days following the subject's exposure to one or more low-level blasts. In some embodiments, the PDE5 inhibitor is administered to the subject daily for a period of at least seven days. In some embodiments, administration of the PDE5 inhibitor to the subject is initiated during a period in which the subject is without chronic TBI. In some embodiments, administration of the PDE5 inhibitor to the subject concludes during a period in which the subject is without chronic TBI.

In some embodiments, the first dosage of the PDE5 inhibitor is administered to the subject within six months of being exposed to one or more low-level blasts. In some embodiments, the first dosage of the PDE5 inhibitor is administered to the subject within three months of being exposed to one or more low-level blasts. In some embodiments, the first dosage of the PDE5 inhibitor is administered to the subject within seven days of being exposed to one or more low-level blasts. In some embodiments, the first dosage of the PDE5 inhibitor is administered to the subject within 24 hours of being exposed to one or more low-level blasts. In some embodiments, the first dosage of the PDE5 inhibitor is administered to the subject within about one hour of being exposed to one or more low-level blasts. In some embodiments, the first dosage of the PDE5 inhibitor is administered to the subject within about 15 minutes of being exposed to one or more low-level blasts.

Treatment via PDE5 inhibitor administration can, in various embodiments, be provided for TBI resulting from a subject's exposure to a single low-level blast or to multiple low-level blasts. In some embodiments of the method for treating low-level blast injury, the subject is exposed to multiple low-level blasts. In some embodiments, the subject is exposed to multiple low-level blasts prior to administration of the PDE5 inhibitor. In some embodiments, a first administration of the PDE5 inhibitor is administered following a first low-level blast, and a second administration of the PDE5 inhibitor is administered following a second low-level blast occurring after the first administration of the PDE5 inhibitor. In some embodiments, the PDE5 inhibitor is first administered to the subject within 24 hours following exposure to a first-occurring blast of multiple low-level blasts to which the subject is exposed. In some embodiments, the PDE5 inhibitor is first administered to the subject following exposure to a last-occurring low-level blast of multiple low-level blasts to which the subject is exposed. In various embodiments, PDE5 is administered to the subject within six months, within five months, within four months, within three months, within two months, within one month, within 14 days, within seven days, within 24 hours, within 1 hour, or within 15 minutes of the last-occurring low-level blast.

In some embodiments of the method for treating low-level blast TBI, the PDE5 inhibitor administered is sildenafil. Tadalafil, vardenafil, avanafil, lodenafil, udenafil, and microdenafil have also been demonstrated to effectively inhibit PDE5, and, in this regard, thus provide the same mechanism of action as sildenafil. As such, embodiments in which the PDE5 administered to the subject following blast exposure is tadalafil, vardenafil, avanafil, lodenafil, udenafil, or microdenafil are also contemplated herein.

Administration of a PDE5 inhibitor has been found to increase levels of cyclic guanosine monophosphate (cGMP), a byproduct of guanylyl cyclase stimulation by nitric oxide, in the brain capillaries of subjects following low-level blast exposure. Nitric oxide (NO) is an important regulator of neurovascular disruption following blast injury. As NO levels may not be sufficient following exposure to one or more low-level blasts, the PDE5 inhibitor can prolong cGMP increase and extend NO-dependent vasodilation, thereby promoting capillary mitochondrial function. cGMP influences proliferator-activated receptor gamma coactivator 1-alpha (PGC-1α) through protein kinase G (PDKG) and downstream transcriptional pathways. PDE5 inhibitor administration has thus further been discovered to increase PGC-1α, a master regulator of mitochondrial biogenesis. As such, and without wishing to be bound by any particular theory, it is believed that PDE5 inhibition effectuated by the administration of PDE5 inhibitor administration, and the increases in cGMP and PGC-1α resulting therefrom, facilitates mitochondrial restoration following mitochondrial biogenesis, thus overcoming oxidative stress and mitochondrial dysfunction by low-level blast exposure. Increases in glial fibrillary acidic protein (GFAP), an astrocyte marker, have also been discovered following low-level blast exposure treated via PDE5 inhibitor administration, indicating that PDE5 inhibition serves to correct astrocyte deficit following blast injury. PDE5 inhibitor administration has also been discovered to increase translocase of the outer mitochondrial membrane complex subunit 20 (TOM20) following low-level blast exposure, indicating that PDE5 inhibitor administration improves mitochondrial density following low-level blast exposure. PDE5 inhibitor administration has also been discovered to increase zonula occludens-1 (ZO-1) following low-level blast exposure, indicating that PDE5 inhibitor administration can improve tight junction integrity following low-level blast exposure.

Accordingly, in another aspect, the presently disclosed subject matter includes a method for mitigating the effects of blast injury on the vascular integrity of a subject in which an effective amount of a PDE5 inhibitor is administered to a subject following the subject's exposure to one or more low-level blasts to thereby increase at least one of capillary respiration, mitochondrial respiration, mitochondrial density, mitochondrial biogenesis, astrocyte level or homeostasis, and tight junction protein expression in the brain of a subject. In some embodiments, an increase in capillary respiration and/or mitochondrial respiration is characterized by an increase in cGMP in the subject relative to one or more non-injured controls, the subject prior to blast injury, or accepted value or range of values corresponding to cGMP levels of healthy controls. In some embodiments, an increase in mitochondrial density is characterized by an increase in TOM20 in the subject relative to one or more non-injured controls, the subject prior to blast injury, or accepted value or range of values corresponding to TOM20 levels of healthy controls. In some embodiments, an increase in mitochondrial biogenesis is characterized by an increase in PGC-1α in the subject relative to one or more non-injured controls, the subject prior to blast injury, or accepted value or range of values corresponding to PGC-1α levels of healthy controls. In some embodiments, an increase in astrocyte level or homeostasis is characterized by an increase in GFAP in the subject relative to one or more non-injured controls, the subject prior to blast injury, or accepted value or range of values corresponding to GFAP levels of healthy controls. In some embodiments, an increase in tight expression is characterized by an increase in ZO-1 in the subject relative to one or more non-injured controls, the subject prior to blast injury, or accepted value or range of values corresponding to ZO-1 levels of healthy controls.

In various embodiments of the method for mitigating the effects of blast injury on the vascular integrity of a subject, the subject can be exposed to a single blast or multiple blasts. Furthermore, in various embodiments of the method for mitigating the effects of blast injury on the vascularity of a subject, the PDE5 inhibitor can be the same, and administered in the same manner, as the various embodiments of the method for treating low-level blast TBI described above.

Administration of PDE5 inhibitor has also surprisingly been discovered to induce a metabolic change in a subject following low-level blast exposure. Specifically, it has been discovered that PDE5 inhibitor administration following low-level blast exposure can reduce nicotinamide levels and drive increased NAD⁺/NADH metabolism. NAD⁺ is essential for mitochondrial oxidative metabolism. Enhancing NAD⁺ availability promotes the activity of key enzymes like sirtuins (particularly SIRT1 and SIRT3), which regulate mitochondrial biogenesis and endothelial function. Additionally, improved NAD⁺/NADH balance supports mitochondrial respiration in endothelial cells, which stabilizes energy production and reduces the production of reactive oxygen species (ROSs). This improvement in mitochondrial efficiency within the brain capillaries guards against oxidative stress that can contribute to both BBB breakdown and neuroinflammation following blast exposure. Furthermore, it is believed that by improving endothelial mitochondrial function and reducing oxidative burden, PDE5 inhibitor-induced enhancement of NAD⁺ metabolism can mitigate the pathological metabolic and inflammatory cascades that sustain post-traumatic stress disorder (PTSD) symptoms following TBI.

Accordingly, in another aspect, the present disclosure also includes a method for inducing a metabolic change in a subject in which an effective amount of a PDE5 inhibitor is administered to the subject following the subjects exposure to one or more blasts to thereby increase NAD⁺/NADH metabolism in the subject. In some embodiments, increased NAD⁺ metabolism is characterized by a decrease in nicotinamide, an increase in NAD⁺, and/or an increase in NADH in the subject relative to one or more non-injured controls, the subject prior to blast injury, or accepted value or range of values corresponding to nicotinamide, NAD⁺, and/or NADH levels of healthy controls.

In various embodiments of the method for inducing a metabolic change in a subject, the subject can be exposed to a single blast or multiple blasts. Furthermore, in various embodiments of the method for a metabolic change in the subject, the PDE5 can be the same, and administered in the same manner, as the various embodiments of the method for treating low-level blast TBI and method for mitigating the effects of blast injury on the vascular integrity of a subject as described above.

In some embodiments of the method for treating low-level blast TBI, the method for mitigating the effects of blast injury on the vascular integrity of the subject, and the method for inducing a metabolic change in a subject, the methods further include of step of identifying a subject as being in need of treatment for blast injury. In some embodiments, the identification of a subject in need of treatment via PDE5 inhibitor administration includes: obtaining a biological sample from a subject that includes one or more cells; assaying the biological sample to detect an expression level or activity of one or more biomarkers in the one or cells of the biological sample selected from cGMP, PGC-1α, GFAP, TOM20, ZO-1, nicotinamide, NAD⁺, and NADH; detecting a measurable difference between the expression or activity level of the one or more biomarkers from the biological sample and a control expression level or activity of the one or more biomarkers; and identifying the subject as being in need of treatment based on the measurable difference. In some embodiments, the measurable difference corresponds to a decrease in cGMP, a decrease in PGC-1α, a difference in GFAP, a decrease in TOM20, a decrease in ZO-1, an increase in nicotinamide, a decrease in NAD⁺, and/or a decrease NADH in expression level or activity in the biological sample relative to a control expression level or activity of such biomarker(s), and the subject is identified as being in need of treatment based on such measurable difference. As reflected in the disclosures that follow, decreases in GFAP have been observed following blast-induced TBI in biological samples obtained from subjects' brains. It has been found, however, that GFAP is increased in the blood following TBI. Accordingly, the difference in GFAP expression level or activity can, in some embodiments, correspond to a decrease in GFAP expression level or activity, and, in other embodiments, correspond to an increase in GFAP expression level or activity, depending on the nature of the biological sample tested.

Accordingly, in yet another aspect, a method for identifying a subject as being in need of blast treatment is also provided herein that includes: obtaining a biological sample from a subject that includes one or more cells; and assaying the biological sample to detect an expression level or activity of one or more biomarkers in the one or cells of the biological sample selected from cGMP, PGC-1α, GFAP, TOM20, ZO-1, nicotinamide, NAD⁺, and NADH; detecting a measurable difference between the expression or activity level of the one or more biomarkers from the biological sample and a control expression level or activity of the one or more biomarkers; and identifying the subject as being in need of treatment based on the measurable difference. In some embodiments, the measurable difference corresponds to a decrease in cGMP, a decrease in PGC-1α, a decrease in GFAP, a decrease in TOM20, a decrease in ZO-1, an increase in nicotinamide, a decrease in NAD⁺, and/or a decrease NADH in expression level or activity in the biological sample relative to a control expression level or activity of such biomarker(s), and the subject is identified as being in need of treatment based on such measurable difference.

With regard to the step of providing a biological sample from the subject, the term “biological sample” as used herein refers to any body fluid and/or tissue potentially comprising the one or more biomarkers described for use herein. In some embodiments, for example, the biological sample can be a blood sample, a serum sample, a plasma sample, or sub-fractions thereof. In some embodiments, the biological sample can be a urine sample. In some embodiments, the biological sample comprises tissue acquired from the subject. In some embodiments, the biological sample comprises multiple biological samples that are assayed for biomarker expression level or activity.

A “biomarker” is a molecule useful as an indicator of a biologic state in a subject. With reference to the present subject matter, the biomarkers disclosed herein can be nucleotides, polypeptides, or metabolites that exhibit a change in expression level or activity, which can be correlated with low-level blast TBI. In addition, the biomarkers disclosed herein corresponding to polypeptides are inclusive of messenger RNAs (mRNAs) encoding the biomarker polypeptides, as measurement of a change in expression of an mRNA can be correlated with changes in expression of the polypeptide encoded by the mRNA. As such, determining an amount of a polypeptide biomarker in a biological sample is inclusive of determining an amount of a polypeptide biomarker and/or an amount of an mRNA encoding the polypeptide biomarker either by direct or indirect (e.g., by measure of a complementary DNA (cDNA) synthesized from the mRNA) measure of the mRNA.

In some embodiments, multiple determinations of one or more biomarkers can be made, and a temporal change in the marker can be used to identify whether a subject is in need of treatment for blast injury. For example, a biomarker can be determined at an initial time, and again at a second time. In such embodiments, an increase or decrease in the biomarker from the initial time to the second time can be indicative of a subject's need for treatment.

Various methods, techniques, and assays for identifying nucleotides, proteins, and metabolites are known in the art and can be employed to identify and quantify the biomarkers disclosed herein. For instance, by way of non-limiting example, in some embodiments, enzyme-linked immunosorbent assay (ELISA), Luminex, FACs, Western blot, dot blot, immunoprecipitation, immunohistochemistry, immunocytochemistry, immunofluorescence, immunodetection methods, optical spectroscopy, radioimmunoassay, mass spectrometry, HPLC, qPCR, RT-qPCR, multiplex qPCR, SAGE, RNA-seq, microarray analysis, FISH, MassARRAY technique, and combinations thereof.

The analysis of biomarkers can be carried out separately or simultaneously with additional markers within one test sample. For example, several markers can be combined into one test for efficient processing of multiple samples and for potentially providing greater diagnostic and/or prognostic accuracy. In addition, one skilled in the art would recognize the value of testing multiple samples (for example, at successive time points) from the same subject. Such testing of serial samples can allow the identification of changes in marker levels over time. Increases or decreases in marker levels, as well as the absence of change in marker levels, can provide useful information about the disease status that includes, but is not limited to, identifying the approximate time from onset of the event, the presence and amount of salvageable tissue, the appropriateness of drug therapies, the effectiveness of various therapies, and identification of the subject's outcome, including risk of future events.

The analysis of markers can be carried out in a variety of physical formats as well. For example, the use of microtiter plates or automation can be used to facilitate the processing of large numbers of test samples. Alternatively, single sample formats could be developed to facilitate immediate treatment and diagnosis in a timely fashion, for example, in ambulatory transport or emergency room settings.

The practice of the presently-disclosed subject matter can employ, unless otherwise indicated, conventional techniques of cell biology, cell culture, molecular biology, transgenic biology, microbiology, recombinant DNA, and immunology, which are within the skill of the art. Such techniques are explained fully in the literature. See e.g., Molecular Cloning A Laboratory Manual (1989), 2nd Ed., ed. by Sambrook, Fritsch and Maniatis, eds., Cold Spring Harbor Laboratory Press, Chapters 16 and 17; U.S. Pat. No. 4,683,195; DNA Cloning, Volumes I and II, Glover, ed., 1985; Oligonucleotide Synthesis, M. J. Gait, ed., 1984; Nucleic Acid Hybridization, D. Hames & S. J. Higgins, eds., 1984; Transcription and Translation, B. D. Hames & S. J. Higgins, eds., 1984; Culture Of Animal Cells, R. I. Freshney, Alan R. Liss, Inc., 1987; Immobilized Cells And Enzymes, IRL Press, 1986; Perbal (1984), A Practical Guide To Molecular Cloning; See Methods In Enzymology (Academic Press, Inc., N.Y.); Gene Transfer Vectors For Mammalian Cells, J. H. Miller and M. P. Calos, eds., Cold Spring Harbor Laboratory, 1987; Methods In Enzymology, Vols. 154 and 155, Wu et al., eds., Academic Press Inc., N.Y.; Immunochemical Methods In Cell And Molecular Biology (Mayer and Walker, eds., Academic Press, London, 1987; Handbook Of Experimental Immunology, Volumes I-IV, D. M. Weir and C. C. Blackwell, eds., 1986.

The presently disclosed subject matter is further illustrated by the following specific but non-limiting examples. Some of the following examples may include aspects that are prophetic, notwithstanding the numerical values, results and/or data referred to and contained in the examples. Further, the following examples may include compilations of data that are representative of data gathered at various times during the course of development and experimentation related to the present invention.

### EXAMPLES

It has been found that a sub-concussive blast (<4 pounds per square inch (PSI)) is unlikely to result in measurable differences in blood-based biomarkers. Low-level blasts (LLBs) in the sub-concussive range (<4 PSI) may thus not result in long-term neuropsychological effects. However, it is not known if the number of or interval between repeated exposures can induce chronic neurological effects.

Distinction between PTSD and blast-induced mild TBI (mbTBI) has historically been difficult as the syndrome and exposures cannot be easily isolated in those affected, such as soldiers and Veterans. Animal models have demonstrated that LLB exposure leads to increases in anxiety, enhanced conditioned fear, in conjunction with dysregulation of amygdala-related fear-mediating proteins. It is established that rats exposed to three repeated LLB (24 hour (h) interval at 11 pounds per square inch (PSI)) produce acute and chronic anxiety and PTSD-related traits. LLB exposure alone can result in persisting behavioral impairment. In addition, this concept has been further explored to show that external stressors, such as predator scents, will induce chronic behavioral change in rats previously given repeated LLB exposure. Thus, a delayed but persistent anxiety response may exist following LLB exposure.

The neurovasculature is selectively vulnerable to blast injury. Vascular pathology occurs acutely but chronic changes have also been observed months after repeated LLB exposure. In one case, it was found that two repetitive blasts initiated acute BBB disruption in the frontal cortex and hippocampus. It has been shown that primary blast exposure (i.e. overpressure exposure, an exposure resulting from the sudden, rapid increase in air pressure above normal atmospheric levels) can cause BBB disruption, leading to a myriad of molecular cascades. BBB disruption can be a primary or secondary pathology after blast exposure. After TBI, this relationship is cyclical due to the finding that BBB disruption is biphasic. Additionally, in some models of blast exposure, BBB impairment is transient, with impairment at six hours after injury before a resolve by 30 days after blast. However, this is observed in models with a single blast exposure and additional studies have shown that repeated LLB exposure results in persisting neurovascular changes, a finding unique to repeated LLB. Although gross BBB changes, such as microcontusion, are reported to only occur with blast exposure over 200 kilopascals (kPa) or 29 PSI peak overpressure, persistent microvasculature impairment is observed after LLB. Indeed, repeated blast exposure (three or more) results in BBB microlesions in the cerebellum.

A unique aspect of the BBB and neurovasculature is the many cell types that regulate the integrity of the neurovascular unit. These cells typically are multi-functional, also regulating neuronal signaling; chief among these are astrocytes. Astrocytes play a large role in BBB health, as end-feet coverage is crucial for proper function and regulation of the BBB. Clinically, compromise of astrocytic endfeet coverage of blood vessels in the brain has been reported in depressive disorders. Disruption of brain microvasculature also impairs the ability for astrocytes to migrate, which could lead to aberrant astrocytosis in regions of blast-induced pathology. The particular parameters, such as the time interval between blast exposures, in repeated LLB exposure leading to neurovascular dysfunction is, however, unknown.

The amygdala is involved in emotional responses, especially in fear and fear conditioning. These responses are characterized by freezing, release of stress hormones, and changes in blood pressure and heart rate which are elicited by activation of the autonomic and hormonal systems. Damage to this system can potentially initiate the neuropsychological cascades in conditions such as PTSD, epilepsy, depression disorders, and TBI. In some neurological disease, such as TBI and PTSD, exacerbation of fear and anxiety through amygdalar hyperactivation occurs. For service members and Veterans with PTSD, activity in the amygdala has been shown to positively correlate with PTSD symptom scores. PTSD is often accompanied by symptoms such as anxiety. Prevalence of self-reported and clinician-rated PTSD symptoms has been found to be higher in Veterans with a history of TBI. Smaller amygdala volume has also been found to be associated with PTSD in Veterans.

In view of the above, and due to prevalence of occupational blast exposure in Veterans, the studies underlying the current examples were carried out to establish a pathological link to the chronic PTSD-like traits observed after repeated LLB exposure, further characterize neurovascular dysfunction occurring after LLB exposure, and to identify treatments for attenuating LLB injury.

### Example 1: Effect of LLB on Behavior and Vascular Integrity

### Methods and Materials

Statistical Analysis. Data analysis for western blot and immunohistological outcomes employed one-way ANOVA or t-test to establish differences in treatment effects since all measures result in parametric data. When warranted post hoc analysis was employed to determine differences in individual group means for the parameter assessed. For recognition index assessment, one sample t-test was performed to compare each group to 0.5, which represent random chance. Significance set a p<0.05 for all analyses.

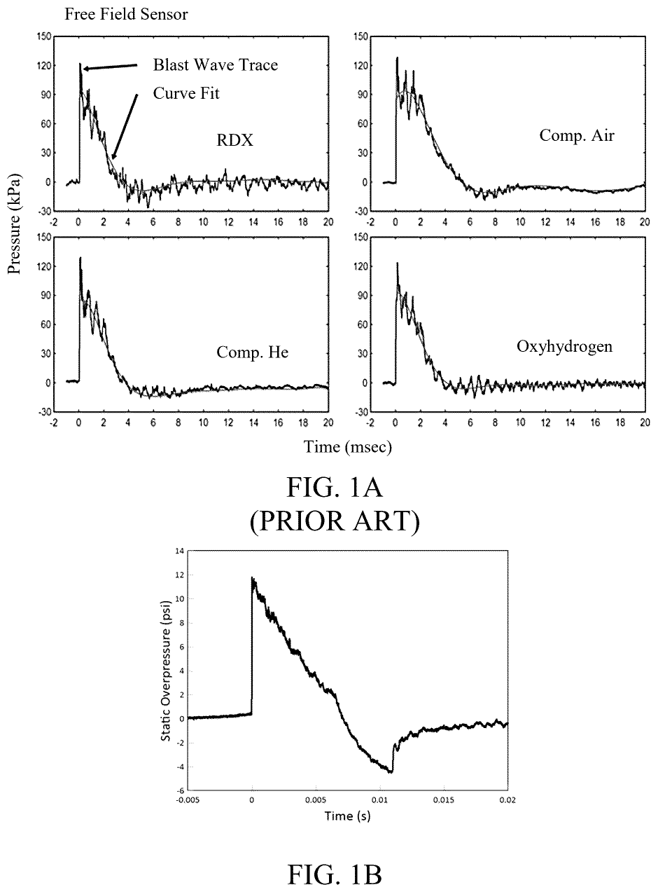

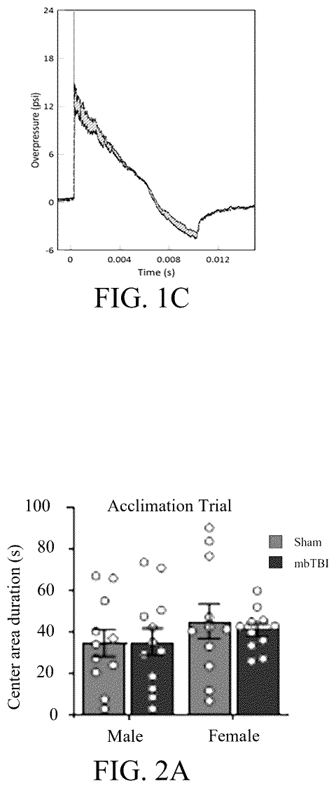

Blast Exposure. A multi-modal blast simulator, termed the McMillan blast device (MBD), that can simulate blast waves using a variety of modalities (FIG. 1A) was utilized to induce blast injury. The MBD consists of a cylindrical steel tube, 12-inch internal diameter, separated into a 19-ft. expansion chamber and a 2.5-ft compression chamber. This blast device can be operated in various modalities using compressed air, compressed helium, oxyhydrogen, or RDX. Compressed helium was utilized as the blast driver to provide experimental consistency and replicates blast curves experienced in military operations, not producing a plateau effect at peak static overpressure

In compressed air- or compressed helium-driven mode, a 10-mil-thick (0.254 mm) biaxially-oriented polyethylene terephthalate (Mylar) membrane separated the two chambers (Mylar A; Tekra Corp., New Berlin, WI). The Mylar membrane was actively ruptured by a 4-point blade affixed to a pneumatic cylinder or passively ruptured at membrane breaking point to achieve desired static overpressure. In the oxyhydrogen-driven mode, a steel manifold was placed between the expansion and compression chambers. A thin polyethylene bag was attached to the manifold and filled with a 2:1 mixture of gaseous hydrogen and oxygen. The oxyhydrogen mixture was ignited by a small cordite charge (Winchester Ammunition #209 ShotShell Primer; Olin Corporation, Clayton, MO).

The shock wave was recorded by face-on, reflected pressure (PCB model #113A24; PCB Piezotronics, Inc., Depew, NY) (FIG. 1B), and free-field averaging sensors (model #137A22) (FIG. 1C). The free-field sensor measuring static overpressure was positioned inside the shock tube with the sensing element located 10 inches from the open end and sensor facing towards the side of the blast tube. The face-on sensor was located inside the blast tube, 10 inches from the open end of the expansion chamber, facing the blast source.

During shockwave velocity recordings, two free-field sensors were positioned inside the tube, separated by a distance of 12 inches. This allowed us to additionally calculate wave speed, determined by time difference between the shock fronts and distance between the sensors. Data from each sensor was routed directly to a TMX-18 (Astronova, Inc.). The rat subjects were secured in plastic netting to minimize but allow free body motion without neck flexion. The rat subjects were positioned 10 inches inside the tube and with lateral head exposure. An overpressure of 11 PSI, which is in the range where neurological disruption has been observed in operations involving breaching activity, was utilized. As this static overpressure is below the threshold to produce lung injury, body shielding was not necessary. To induce mild blast traumatic brain injury (mbTBI) male and female Sprague Dawley rats were subjected to a single blast. To induce repeated mild blast traumatic brain injury (rmbTBI) male and female Sprague Dawley rats were subjected to either two blasts at 1 hour time intervals or two blasts at 24 hour time intervals (i.e., a single blast per day for two days).

Rats were euthanized seven days after blast exposure and saline perfused followed by hemisection. One hemisphere was fresh frozen for western blot analysis while the other hemisphere was fixed for immunohistology. For western blot, the amygdala and hippocampus were lysed and examined for the markers disclosed herein. Fixed half brains were sectioned coronally and the whole brain and sub-region amygdala were examined using immunostaining.

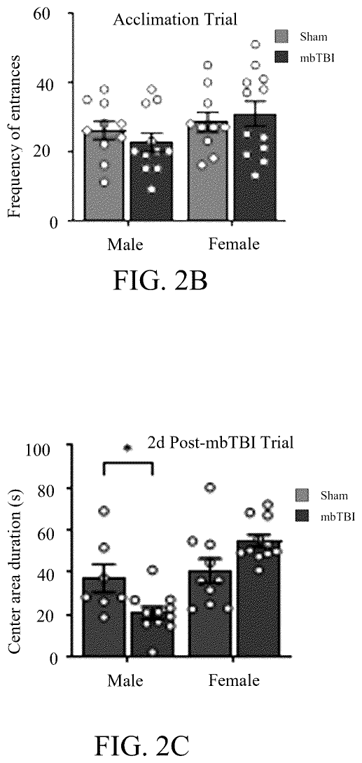

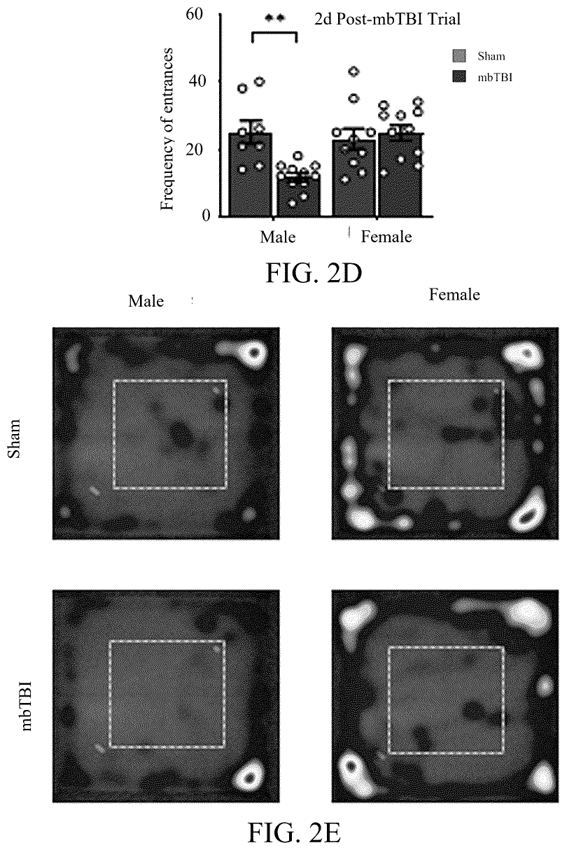

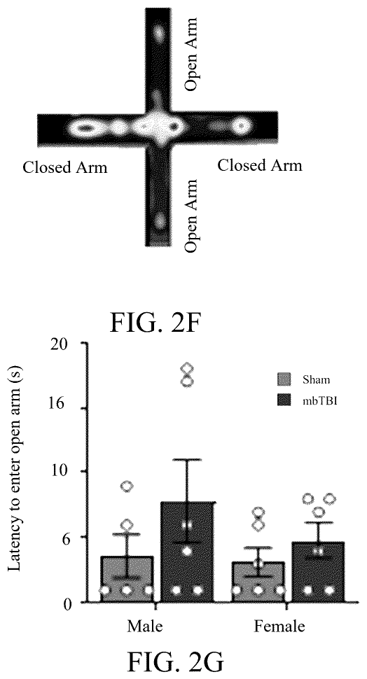

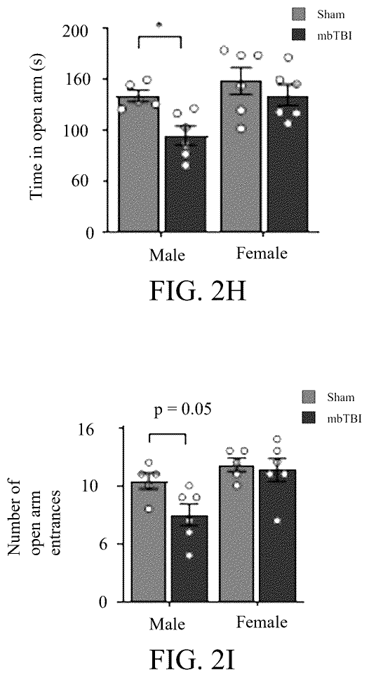

Behavioral Deficits Assessment. The behavioral effects of mbTBI was assessed between male and female sham and mbTBI subjects utilizing an open field test at three days pre-mbTBI and two days post-mbTBI (FIGS. 2A-2E). Rats were placed in a 32″×32″×12″ dimly lit box for 10 min and their exploration was recorded using Ethovision software. Using software, the box was divided into two zones, with the inner zone being half the size and centered within the outer zone. The software tracked the nose point of the rats and recorded the number of entrances and time spent in each zone. The box was cleaned using 70% EtOH between each test. Exclusion criteria was set based on baseline performance in the acclimation trial (<10% time in center area). Elevated plus maze was performed at seven days post-mbTBI treatment (FIGS. 2F-2I). The maze is designed with two open arms, 20″×4″, and two closed arms, 20″×4″×15.94″, with like arms across from each other and an open junction in the middle, 4″×4″. The plus maze has no roof and raised off the ground 29.31″. Rats were placed in the junction between the open and closed arms and allowed to explore for five minutes. The number of entrances into and time spent in either the closed or open arms was recorded using IR beam detection by MedPC software. The lights were slightly dimmed and the box was cleaned between each trial using 70% EtOH.

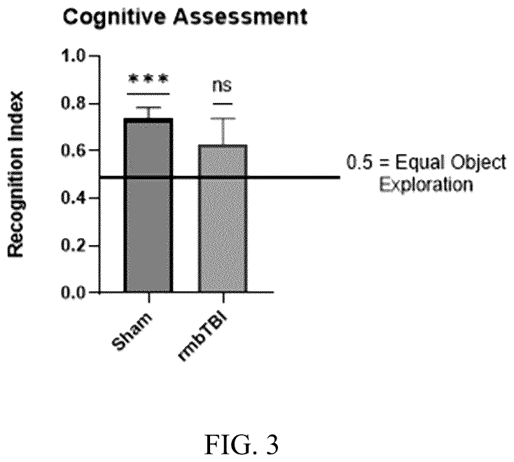

Novel Object Recognition Task. Novel object recognition task was used in rodent models to assay cognitive performance following mbTBI. The first trial involved the exposure of the animal to identical “familiar” objects for five minutes. In the second trial, animals were exposed to a “familiar” object (same object used in the first task) and a “novel” object for five minutes. Trials took place in an opaque black acrylic box with dimensions 80×80×36 cm and animal behavior was tracked using Etho Vision XT™ tracking software. Precautions were taken to clean the chamber between the trials and have the experimenter leave the room during the experiment. For analysis, a recognition index was calculated for each trial (time spent exploring the familiar object relative to the novel object divided by total time exploring objects during each trial). A ratio of 0.5 indicated equal exploration of both objects during the trial. Results were assessed one week after blast exposure (FIG. 3).

Neurovascular Integrity Assessment. Blood-brain barrier (BBB) integrity was assessed using SMI-71 following blast exposure in whole brain sections and amygdala regions of male and female sham and mbTBI subjects 24 hours and 7 days post-mbTBI. SMI-71, which stains endothelial barrier antigen (EBA) and is a marker for intact BBB, has been correlated with FITC-albumin infiltration. Lower numbers of EBA+ vessels and stained vessel area are associated with regions of BBB dysfunction.

Astrocyte, Pericyte, Microglial, and Tight Junction Protein Assessment. Levels of aquaporin-4 (AQP4), indicative of astrocytic end-feet at the BBB, were measured via AQP4 immunofluorescence in the cortex of male and female rats at seven days following rmbTBI-1 hr (2 blasts at 1 h interval) or rmbTBI-24 hr (2 blasts at 24 h interval). Brain sections were permeabilized and blocked using a solution containing 0.2% Triton X-100 in PBST, 1% BSA, and 10% normal horse serum for 1 hour at room temperature (RT). Following blocking, samples were incubated overnight at 4° C. with primary antibody, Aquaporin-4 (AQP4) (59678S Cell Signaling. Secondary antibodies were used in 1:500 dilution at 1 h RT. Samples were mounted on glass slides using a Vectashield HardSet Antifade Mounting Medium with DAPI (H-1500-10; Vector laboratories). Images were acquired using a Nikon confocal microscope with NIS-Elements version 5.30.05.

GFAP and SMI-71 double immunostaining was further employed to assess astrocyte coverage around the brain capillary from amygdala of male and female sham and mbTBI subjects. Randomly selected (n=3-6/group) above mentioned brain sections were double immuno-stained for GFAP (1:250; G9269, Sigma) and SMI-71 (1:250; 836804, Biolegend) or SMI-71 alone to quantify the astrocyte coverage around the brain vasculature and BBB integrity respectively. Briefly, brain sections were permeabilized in 0.2% Trion X-100 in TBS for 15 mints followed by blocking in blocking buffer (1% BSA+10% normal horse serum+0.1% Triton X-100 in TBS) at RT for 1 h. Then sections were incubated with mixture of rabbit anti-GFAP and mouse anti-SMI-71 or SMI-71 primary antibody in blocking buffer overnight at 4° C. Following day, sections were washed and incubated with mixture of Alexa flour 488 donkey anti-rabbit (1:500; A212206, Invitrogen) and Alexa flour 594 donkey anti-mouse (1:500; A212203, Invitrogen) or Alexa flour 594 donkey anti-mouse alone as secondary antibody in blocking buffer at room temperature (RT) for 1 h. After rinsing, samples were mounted on glass slides using prolong-glass antifade mount with Nucblue (P36981; Invitrogen). GFAP and SMI-71 double stained slides were scanned and astrocytic end-feet coverage around the blood vessel were analyzed using Nikon confocal microscope (20×; 100× with oil) with NIS-Elements version 5.30.05. Randomly 25 vessels were selected (red channel; SMI 71) in amygdala region from each brain sections (n=3/group) and measured GFAP mean intensity (green channel) after subtracting the background fluorescence (Additional file 1). SMI-71 stained slides were scanned using BioTek-Cytation-5 with Gen5 Image+3.11 software. Vascular integrity was quantified as vascular density by SMI-71 using ImageJ with vascular density macro. Western blot densitometry quantification of sham and mbTBI of male rats seven days post-rmbTBI-24 hr was performed using pericyte marker platelet-derived growth factor receptor-β(PDGFR-β), astrocyte marker glial fibrillary acidic protein (GFAP), and microglial marker ionized calcium-binding adaptor molecule 1 (IBA1). Briefly, 150 μL of whole brain homogenate was collected during brain capillary isolation and mixed with 10× RIPA buffer (150 mM NaCl, 1% Triton X-100, 0.5% sodium deoxycholate, 0.1% SDS, 50 mM Tris, pH 8.0) supplemented with protease inhibitors. The samples were snap-frozen in liquid nitrogen and stored at −80° C. until further use. For western blot analysis, lysates were prepared using XT sample buffer (1610791, Bio-Rad) with DTT, boiled at 95° C. for 10 minutes, and resolved on 4-12% BIS-TRIS gels (3450125, Bio-Rad) under reducing conditions. Proteins were transferred onto PVDF membranes and blocked with 5% non-fat dry milk in TBS-T. The membranes were incubated overnight at 4° C. with primary antibodies against GFAP (1:1000, G3893; Sigma), IBA-1 (1:1000, 019-19741; Wako Fujifilm), PDGFR-β (1:1000, 3169S; Cell Signaling), and β-Actin (1:5000, 4970S; Cell Signaling). Fluorescent secondary antibodies IRDye 680RD goat anti-mouse (1:10,000; 926-68070, LI-COR Biotechnology) and IRDye 800CW goat anti-rabbit (1:10,000; 926-32211, LI-COR Biotechnology) were applied for 1 hour at room temperature. Blots were imaged using the LI-COR Odyssey DLx imager (LI-COR Biotechnology, Nebraska, USA). Protein levels were quantified by densitometric analysis using ImageJ software and normalized to β-Actin and presented as fold changes relative to sham.

Tight junction protein assessment was performed utilizing immunofluorescence staining and fluorescence quantification of tight junction protein markers occludin, zonula occludens-1 (ZO-1), and claudin-5 on the capillaries of male and female rats 1 h and 24 h using Nikon software. Isolated capillaries were loaded into an 8-well glass chamber slide and allowed to settle for 1 hour at 25° C. The capillaries were then fixed with 10% neutral buffered formalin for 10 minutes. Capillaries were permeabilized and blocked using a solution containing 0.2% Triton X-100 in PBST, 1% BSA, and 10% normal horse serum for 1 hour at room temperature (RT). Following blocking, samples were incubated overnight at 4° C. with various primary antibodies. All the primary antibodies were used in 1:250 dilution: Zona occludens-1 (ZO-1) (ZO1-1A12; Thermofisher), Occludin-1 (OC-3F10; Thermofisher), Claudin-5 (4C3C2; Thermofisher). All the secondary antibodies were used in 1:500 dilution at 1 h RT: Alexa flour 594 donkey anti-rabbit (A212207; Invitrogen), Alexa flour 488 donkey anti-mouse (A11001; Invitrogen), and Streptavidin, DyLight 649 (SA-5649-1; Vector laboratories). Samples were mounted on glass slides using a Vectashield HardSet Antifade Mounting Medium with DAPI (H-1500-10; Vector laboratories). Images were acquired using a Nikon confocal microscope with NIS-Elements version 5.30.05.

### Results

Anxiety and Behavioral Deficits After Blast Exposure. The duration and frequency of entrances in the center of the open field box for the male mild blast injury group was significantly decreased compared to sham (FIGS. 2A-2D). A representative image of animal activity over the five-minute period in the open arena demonstrates global exploration by the sham group and proximity to the walls in the blast group (FIG. 2E). The light & dark (L&D) box paradigm in a rat model of blast exposure has previously been used to examine anxiety-like behavior. No significant changes were observed between the female sham and mbTBI subjects in the open field tests. No significant change was found with respect to male mbTBI group latency in entering the open arms as compared to sham (FIG. 2G) in the elevated plus maze task (FIG. 2F). However, the male mbTBI group was found to display significantly less open arm time as compared to sham (FIG. 2H) and had a significant decrease in the number of entrances into open arms (FIG. 2I), as compared to sham. Such actions are consistent with anxiety-like behavior and could be the neurological manifestation of an underlying neurovascular pathology resulting from blast exposure. No significant changes were observed between the female sham and mbTBI subjects in the open plus task. Accordingly, the results of the open field test and elevated plus maze thus suggested that mbTBI alters exploratory and anxiety-related behavior in male rats, but not in female rats.

As shown in FIG. 3, one week after blast exposure, in the novel object recognition test, male sham animals were found to explore the novel object (an index of proper cognitive function) at a level significantly more than chance, whereas male rmbTBI groups (2 blasts at 24 h interval) displayed no significant difference compared to chance, thus reflecting a lack of learning or cognitive function. This data demonstrates cognitive dysfunction occurring in rats after rmbTBI.

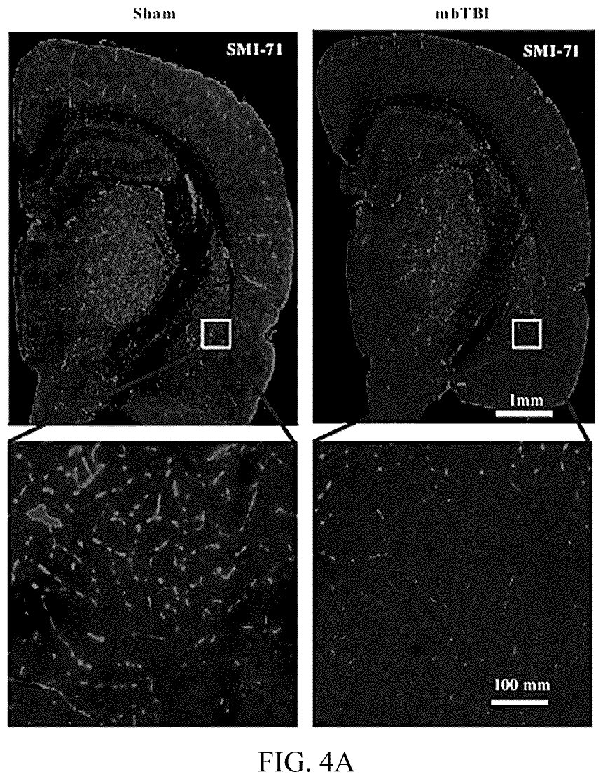

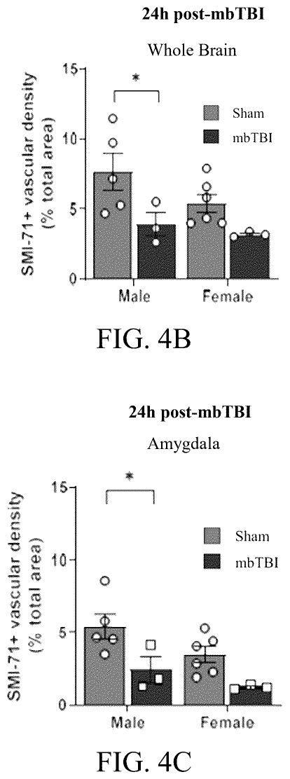

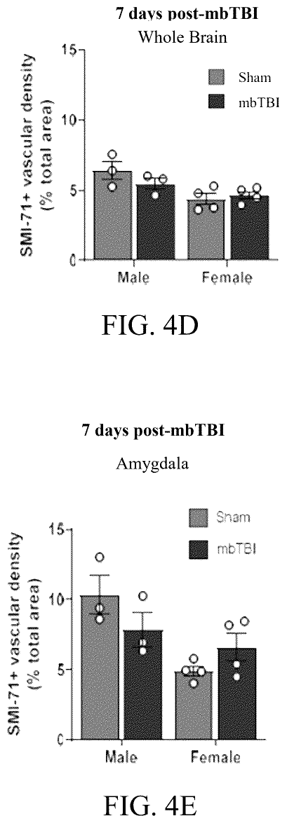

Disruption of Neurovascular Integrity After Blast Exposure. SMI-71 expression was decreased in the male mbTBI group in both the whole brain and amygdala region as compared to the sham group, with a significant decrease being observed 24 h following mbTBI (FIGS. 4A-4E). SMI-71 binds to endothelial barrier antigen (EBA), which is not present in vessels with BBB disruption. The decrease in SMI-71 expression thus signifies decreased vessel count due to EBA expression and compromised BBB in the male mbTBI group. There were lower levels of vascular integrity in females following mbTBI as compared to sham. By seven days post-mbTBI, vascular integrity were not significantly different following mbTBI as compared to sham for both sexes. We show that males experienced a greater degree of acute BBB breakdown in the amygdala and whole brain following mbTBI.

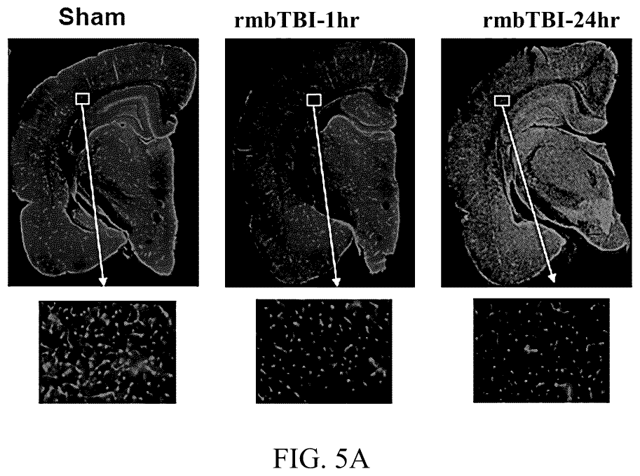

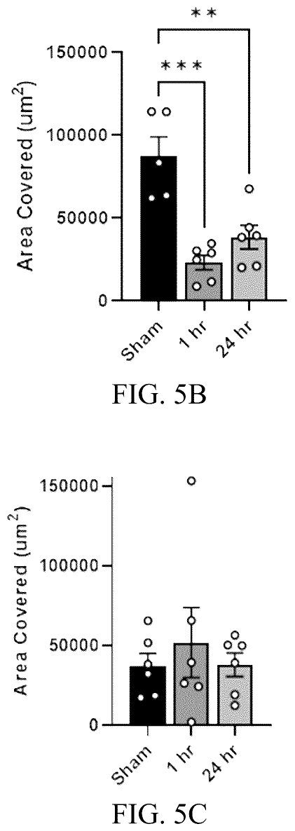

Astrocyte abnormalities as a Hallmark of Blast Exposure. Astrocytic end-feet play a critical role in BBB regulation. Aquaporin-4 (AQP4) levels are indicative of astrocytic end-feet at the BBB. Decreased staining of AQP4 was observed in rmbTBI groups compared to sham group (FIG. 5A). There was a significant quantified decrease in AQP4 levels in male rmbTBI group compared to sham group for both rmb-TBI-1 hr (2 blasts separated by 1 h) and rmbTBI-24 hr (2 blasts separated by 24 h) at 7 days post-rmbTBI (FIG. 5B). A similar decrease was not observed in the female rmbTBI group (FIG. 5C). These findings suggest that rmbTBI induces subacute microvascular injury and BBB disruption in male animals, while females do not exhibit an ongoing vascular response to rmbTBI.

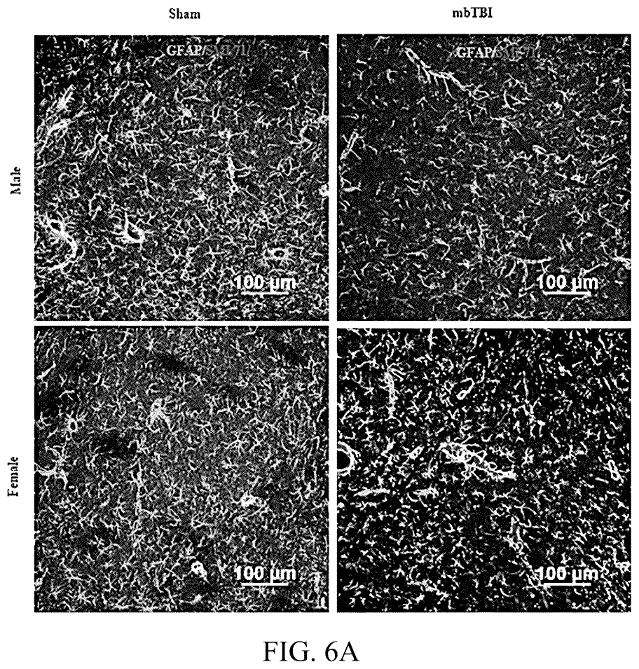

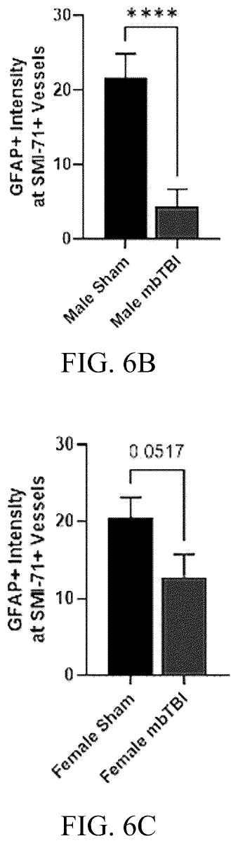

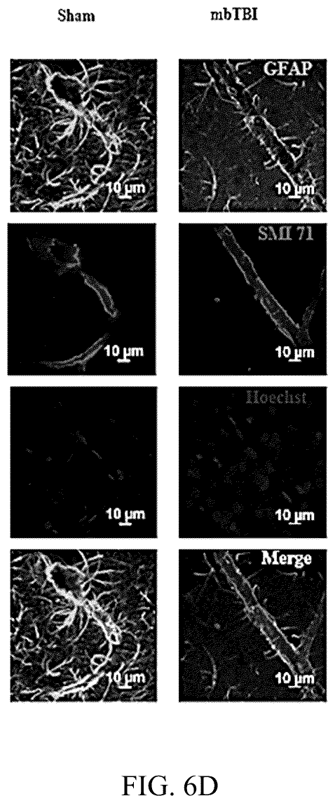

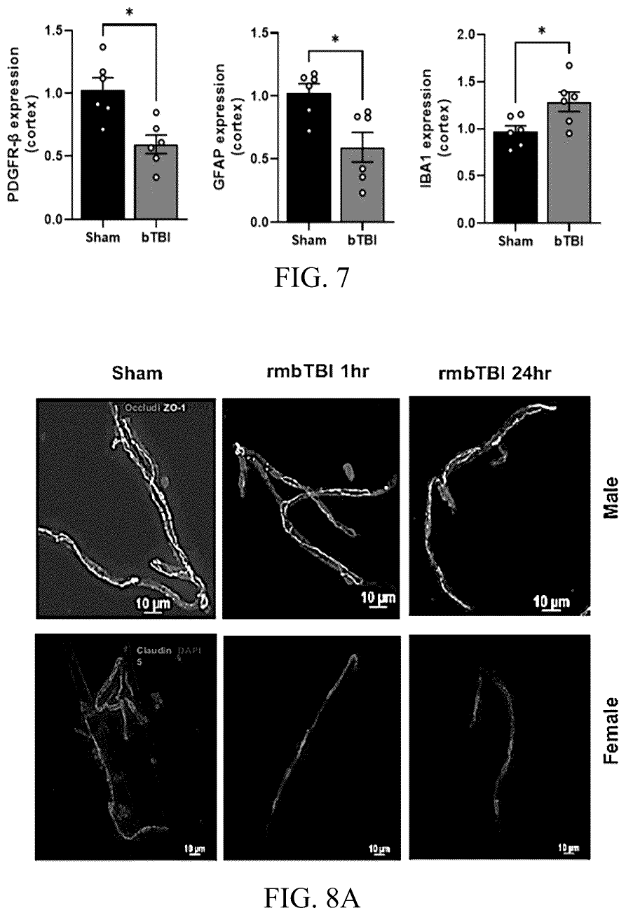

To understand GFAP decreases relative to early pathology following mbTBI, fluorescent co-staining was performed to explain interplay between astrocytes and capillaries. Astrocytic end-feet are tightly intertwined to BBB health. At 24 h post-mbTBI, we observed a decrease in GFAP expression around SMI-71+ vessels in the amygdala of male mbTBI animals. Male mbTBI animals displayed a greater impairment in astrocytic capillary coverage as compared to female mbTBI animals, compared to respective sham groups. High magnification micrographs show the cellular localization in male mbTBI animals at 24 h post-mbTBI (FIGS. 6A-6D). Depression of astrocyte levels (GFAP), lowering pericyte levels (PDGFR-β), and an increase in microglia (IBA1) levels was observed in the cortex region of male rats seven days after rmbTBI (2 blasts separated by 24 h) compared to sham (FIG. 7). These findings suggest that alterations in astrocytic, microglial and pericyte pathology are present in male rmbTBI groups relative to sham.

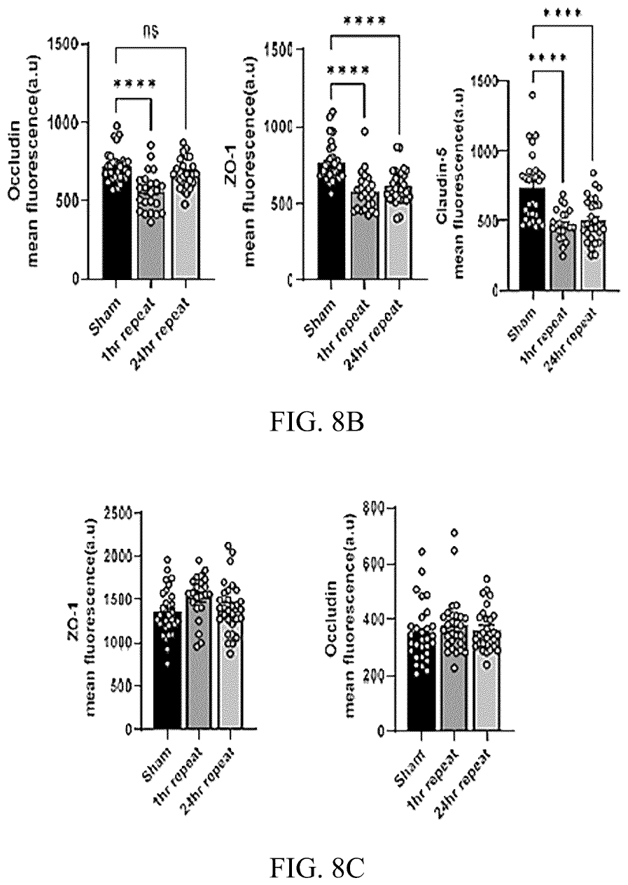

A decrease in tight junction proteins (occludin, ZO-1, claudin-5) was observed in the male rmbTBI group seven days post-rmbTBI at both 1 h and 24 h blast intervals, but not in the female rmbTBI group (FIG. 8A-8C). This indicates a sustained impact of rmbTBI on tight junction integrity in male rats. Contrary to male rats, female rats did not exhibit significant differences in occludin, claudin-5 and ZO-1 fluorescence intensity between sham and rmbTBI groups suggesting a potential sex-specific difference in response to rmbTBI.

### Example 2: Mitochondrial Function in Brain Capillaries with PDE5 Inhibition

### Methods and Materials

Blast Exposure. The MBD consists of a cylindrical steel tube, 12-inch internal diameter, separated into a 19-ft. expansion chamber and a 2.5-ft compression chamber. For these studies, compressed helium-driven mode was used to replicate military blast exposure. A 10-mil-thick (0.254 mm) biaxially-oriented polyethylene terephthalate (Mylar) membrane separates the two chambers. The Mylar membrane was actively ruptured by a 4-point blade affixed to a pneumatic cylinder to achieve desired static overpressure. The shock wave was recorded by face-on, reflected pressure (Kulite pitot-static pressure probe) and wall-mounted side-on static pressure sensors (Kulite). Data from each piezoresistive sensor was routed through sensor cables before being captured by the state-of-the-art TMX-18 (AstroNova™ Test & Measurement, 800 kHz sampling rate) and analyzed using AstroView. The rat subjects were secured in plastic netting to minimize but allow free body motion without neck flexion. The rat subjects were positioned 30 inches inside the tube and with lateral head exposure. An overpressure of 11 PSI was utilized, which is in the range where neurological disruption has been observed in operations involving breaching activity. In the studies underlying this example, Sprague Dawley rats were subjected to single blast exposure.

Capillary Isolation. Brain capillaries were isolated as previously described. Animals were euthanized by CO₂ inhalation and decapitated, brains were removed, dissected, and homogenized in cold PBS buffer (2.7 mM KCl, 1.46 mM KH2PO4, 136.9 mM NaCl, 8.1 mM Na2HPO4, 5 mM D-glucose, 1 mM sodium pyruvate, pH 7.4). Ficoll® was added to the brain homogenate to a final concentration of 15% and the Ficoll®/brain mixture was centrifuged at 5,800 g for 20 min at 4° C. After resuspending the pellet in 1% BSA/PBS, the capillary suspension was passed over a 30 μm filter to purify capillaries from debris and blood cells. Capillaries were washed and used for experiments.

Capillary Respirometry Profiling. Vessel respiration was conducted according to our preliminary study and Sure, et al. (2018). Using a Seahorse XFe96 Flux Analyzer (Agilent Technologies, Palo Alto, CA, USA), the mitochondrial stress test was performed according to the manufacturer's instructions. Briefly, capillaries from each group were centrifuged, and the pellet was resuspended in XF assay medium with substrates (10 mM glucose, 1 mM pyruvate, and 2 mM L-glutamine). Each sample was equally distributed as triplicates in XF microplates and centrifuged at 800×g for 10 min to settle the capillaries at the bottom of the well. After 1 h of incubation at 37° C., the 96-well XF microplate was loaded into an XFe96 Flux analyzer to measure oxygen consumption rate (OCR). After three measurements of baseline OCR (basal respiration), respiratory chain inhibitors/uncouplers were sequentially injected into each well (1 μM Oligomycin, 4 μM FCCP, 0.5 μM Rotenone/Antimycin A, and 5 mM 2-deoxyglucose (2DG)), and OCR was measured three times after each injection. Maximal respiration was calculated on OCR data recorded after FCCP addition and basal glycolysis was calculated on Extracellular Acidification Rate (ECAR) data recorded after 2-DG addition. At the end of the experiment, the XF microplate was centrifuged at 1000 g for 10 min, after removing the media, 10 μl RIPA buffer was added to the capillaries in each well, and freeze-thaw cycles were performed 3 times in dry ice before protein was quantified using a BCA kit (23225; Thermo Scientific). Using Wave software version 2.6.1 (Agilent Technologies, Santa Clara, CA, USA), OCR from various respiration states was calculated and normalized to protein concentration

Respiration in Capillaries Following mbTBI. Capillaries from the rat cortex were isolated according to previous reports and varying amounts of protein (5-25 μg) were aliquoted into wells of a Seahorse XFe96 microplate and the mitochondrial stress test was performed. To examine capillary metabolism following mbTBI, capillary respiration was measured at 1 day post-mbTBI (11 PSI peak overpressure).

Efficacy of Sildenafil in Restoring Vascular Function After mbTBI. Capillaries were isolated from the cortex of female and male Sprague Dawley rats (n=6/group) at 1d after mbTBI. Capillaries of both groups were isolated and bathed in sildenafil citrate solution (100 μg/mL) before Seahorse analysis. Two separate cohorts were studied. Male and female rodents went through sham or mbTBI procedures before brain extraction and capillary isolation at one day following mbTBI. Capillaries were bathed in control (cont) or sildenafil (silde) for two hours before undergoing Seahorse analysis.

Mitochondrial Mechanisms Targeting mbTBI. To further investigate the effect of sildenafil on mitochondrial mechanisms for targeting mild blast TBI, rat brain microvascular endothelial cells incubated with sildenafil (1 μM in 1% DMSO diluted in cell culture media) or vehicle (1% DMSO diluted in cell culture media) overnight were labeled with mitotracker green to examine mitochondrial biogenesis. Mitochondrial Network Analysis (MiNA) workflow in ImageJ was used to examine mitochondrial parameters. These cell culture experiments are considered proof-of-concept that PDE5 inhibition by sildenafil can induce mitochondrial increases.

### Results

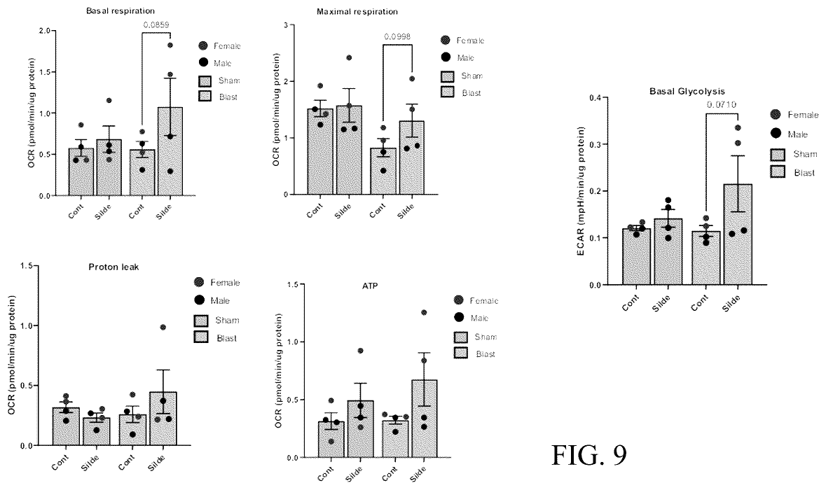

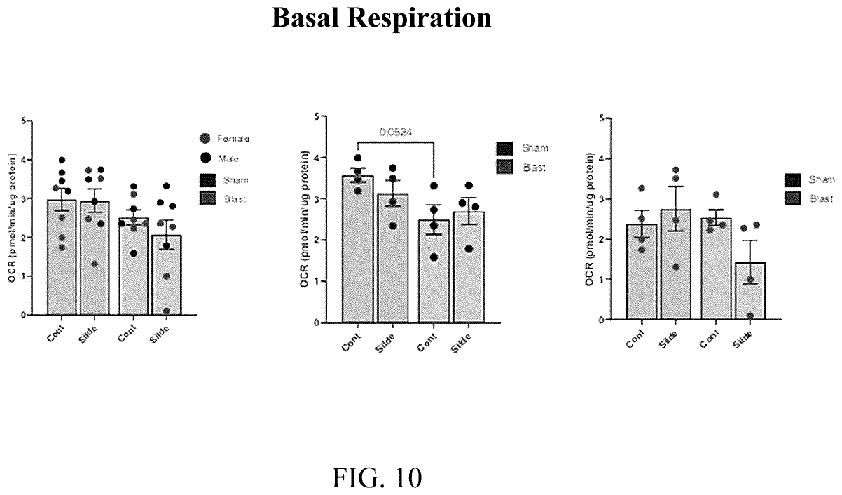

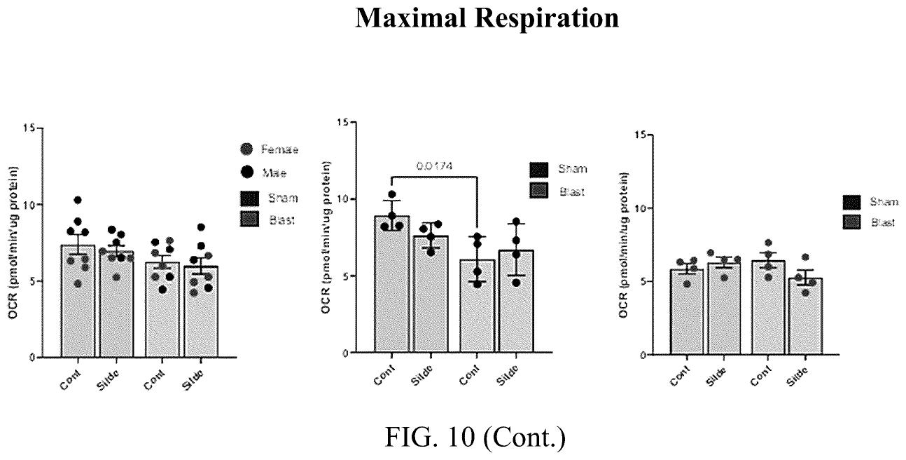

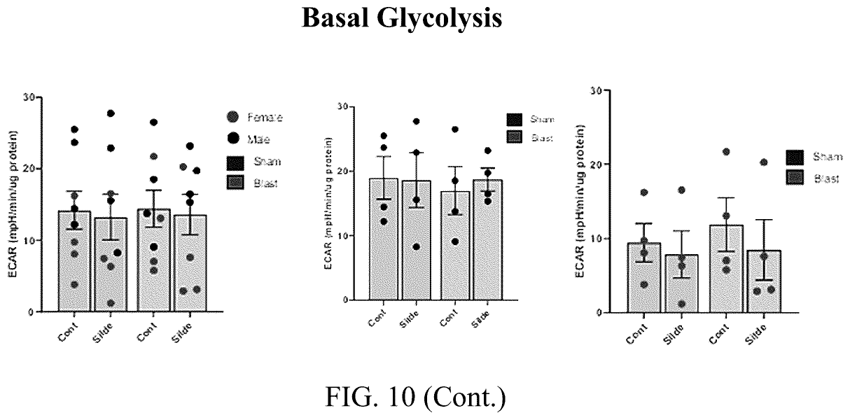

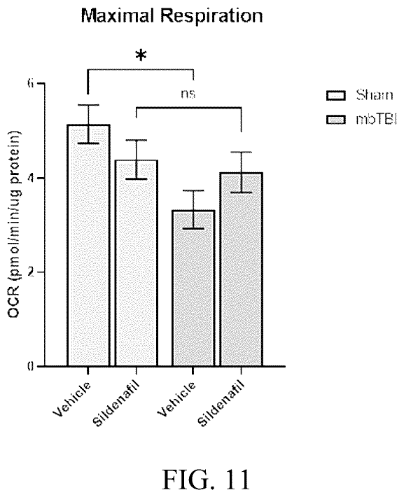

In the first cohort, sildenafil non-significantly increased basal respiration, maximal respiration, and basal glycolysis in mbTBI groups (FIG. 9). In the second cohort of animals, male mbTBI control group displayed a deficit in maximal respiration compared to its respective sham group. Sildenafil administration normalized maximal respiration responses following mbTBI (FIG. 10). Data combined from both cohorts confirmed that sildenafil administration can restore mbTBI-induced deficits in capillary-specific maximal mitochondrial respiration (FIG. 11). These results highlight the ability of sildenafil, and potentially other PDE5 inhibitors, to mitigate capillary specific mitochondrial deficits following mbTBI or exposure to low-level blasts.

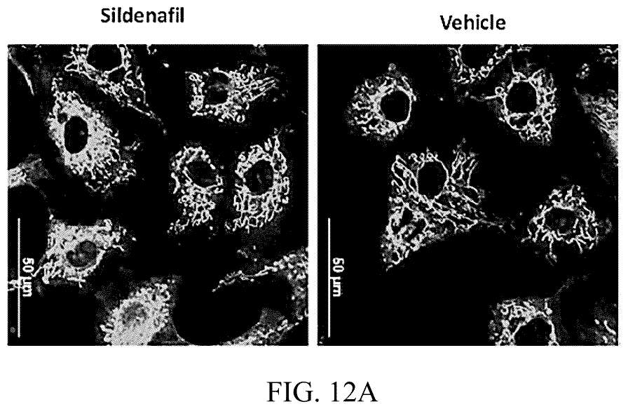

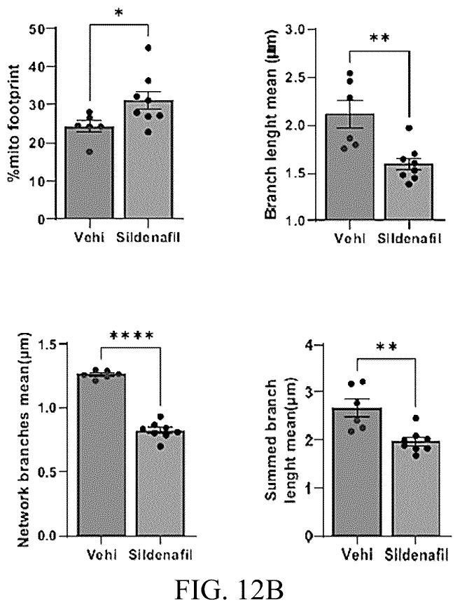

Sildenafil administration increased mitochondrial density compared to vehicle (FIG. 12B). Mitochondrial branching was also found to be decreased after sildenafil compared to vehicle (FIG. 12A), which could be due to biogenesis-mediated mitochondrial fission processes. These data support that the use of PDE5 inhibitors can improve mitochondria morphology to increase metabolism for the treatment of mbTBI.

### Example 3: Sildenafil-mediated Improvement of Mitochondrial Biogenesis and Content in Brain Capillaries Following Repeated Mild Blast Exposure

### Methods and Materials

Blast Exposure. The MBD consists of a cylindrical steel tube, 12-inch internal diameter, separated into a 19-ft. expansion chamber and a 2.5-ft compression chamber. For these studies, compressed helium-driven mode was used to replicate military blast exposure. A 10-mil-thick (0.254 mm) biaxially-oriented polyethylene terephthalate (Mylar) membrane separates the two chambers. The Mylar membrane was actively ruptured by a 4-point blade affixed to a pneumatic cylinder to achieve desired static overpressure. The shock wave was recorded by face-on, reflected pressure (Kulite pitot-static pressure probe) and wall-mounted side-on static pressure sensors (Kulite). Data from each piezoresistive sensor was routed through sensor cables before being captured by the state-of-the-art TMX-18 (AstroNova™ Test & Measurement, 800 kHz sampling rate) and analyzed using AstroView. The rat subjects were secured in plastic netting to minimize but allow free body motion without neck flexion. The rat subjects were positioned 30 inches inside the tube and with lateral head exposure. An overpressure of 11 PSI was utilized, which is in the range where neurological disruption has been observed in operations involving breaching activity. In the studies underlying this example, Sprague Dawley rats were subjected to repeated blast exposure (2 blasts at 24 h interval).

Capillary Isolation. After perfusion, rats were decapitated, and the brains were then removed from the skull. One hemisphere of the brains was rapidly dissected and snap frozen in liquid nitrogen (LN2) for capillary isolation. An aliquot of tissue was chopped into small pieces using a razor blade and transferred into a 2 mL screw-cap tube (Sarstedt Inc Screw Cap Microtube, type H) preloaded with stainless beads (3.2 mm, 1.8 g, fisher scientific, NC0778455). 1 ml of ice-cold capillary isolation buffer was then pipetted into the tube before samples were homogenized in bead homogenizer (Biospec products) for 15 s. Tissue homogenate was transferred into a 5 ml tube. The screw cap tube and beads were rinsed with isolation buffer and transferred to the same 5 ml tube, and isolation buffer was added for final homogenate volume of 5 ml. Homogenate was vortexed quickly and centrifuged at 1000×g for 10 min at 4° C. using a swinging bucket rotor. The supernatant was removed carefully, and 4 mL of lymphocyte separation medium (25-072-CV; Corning Life Sciences) was added to the pellet to separate the brain vessels from myelin and other brain cells by gradient centrifugation. The homogenate was vortexed for 30 s to obtain a homogenous suspension, which was then centrifuged at 4500×g for 20 min at 4° C. using a swinging bucket rotor. The top myelin layer was removed along with the supernatant, and the inner sides of the tube were cleaned to remove leftover myelin debris using Kim Wipes or cotton buds. The pellet was resuspended in 1 ml of ice-cold isolation buffer, and the resulting homogenate was filtered through a 70 μm membrane filter (15-1070; Tisch Scientific) to remove larger vessels. The membrane was then washed with at least 10 ml of isolation buffer. The filtrate was then filtered through an 18 μm membrane (25 mm diameter) filter (ME17233; Tisch Scientific) assembled with a modified the filter holder (SF18128; Tisch Scientific), as shown in FIG. 1A. One 25 mm filter is sufficient to filter capillaries from one mouse cerebrum without clogging of the filter. At least 10 ml of wash buffer (DPBS; Cat No: 14080055, Fisher Scientific with 5.5 mM glucose, 1 mM sodium pyruvate with pH 7.4) was used to wash the membrane to remove RBCs, suspended brain cells and debris. Using tweezers, the 18 μm membrane filter with capillaries on the top was removed from the filter holder and placed inside the 1 ml microcentrifuge tube wall. Using a 1 ml pipette, all the capillaries were eluted from the membrane by flushing 1 ml of wash buffer 2-3× followed by quick vortexing. A drop of capillary elute was used to assess the quality and concentration of the capillaries under a bright field microscope. Final centrifugation was performed at 1000×g, 10 min, 4° C. (swinging bucket) to pellet the isolated brain capillaries.

Tissue Isolation. For downstream western blot, whole brain homogenate was collected during brain capillary isolation and mixed with 10× RIPA buffer (150 mM NaCl, 1% Triton X-100, 0.5% sodium deoxycholate, 0.1% SDS, 50 mM Tris, pH 8.0) supplemented with protease inhibitors. The samples were snap-frozen in liquid nitrogen and stored at −80° C. until further use.

cGMP Concentration. The effect of sildenafil treatment on cyclic guanosine monophosphate (cGMP) following rmbTBI was assessed by comparison of isolated control capillaries and isolated sildenafil-treated capillaries (100 nM, 1 μM, or 10 μM) obtained from rat cortex. Capillaries were isolated from rat cortex and incubated for two hours and then media was collected and tested using cGMP ELISA kit.

Mitochondrial Biogenesis and Astrocyte Homeostasis. The effect of sildenafil treatment on mitochondrial biogenesis and astrocyte homeostasis was assessed by comparing sham subjects administered daily with vehicle, rmbTBI subjects administered daily with vehicle immediately (within about 15 minutes) following each respective blast exposure and continuing daily to day seven, rmbTBI subjects administered daily with low-dose sildenafil treatment (5 mg/kg) immediately following (within about 15 minutes) each respective blast exposure and continuing daily to day seven, and rmbTBI subjects administered daily with high-dose sildenafil treatment (20 mg/kg) immediately (within about 15 minutes) following each respective blast exposure and continuing daily to day seven. Expression of peroxisome proliferator-activated receptor gamma coactivator 1-alpha (PGC-1α), glial fibrillary acidic protein (GFAP), and β-actin was assessed in cortical tissue using western blot. Cortical tissue was extracted and probed seven days post-rmbTBI. Expression of PGC-1a and β-actin was also assessed using in the hippocampal tissue of subjects using western blot. Hippocampal tissue was extracted and probed seven-days post-rmbTBI.

Capillary-Specific Mitochondrial Content. The effect of sildenafil treatment on capillary-specific mitochondrial content was assessed by comparing rmbTBI subjects administered daily with vehicle immediately (within about 15 minutes) following each respective blast exposure and continuing daily to day seven, rmbTBI subjects administered daily with low-dose sildenafil treatment (5 mg/kg) immediately (within about 15 minutes) following each respective blast exposure and continuing daily to day seven, and rmbTBI subjects administered daily with high-dose sildenafil treatment (20 mg/kg) immediately (within about 15 minutes) following each respective blast exposure and continuing daily to day seven. Capillaries were isolated at seven days post-rmbTBI and stained for mitochondria marker translocase of outer mitochondrial membrane 20 (TOM20), isolectin, and DAPI.

Tight Junction Integrity. The effect of sildenafil treatment on tight junction proteins was assessed by comparing sham subjects administered daily with vehicle, rmbTBI subjects administered daily with vehicle immediately (within about 15 minutes) following each respective blast exposure and continuing daily to day seven, rmbTBI subjects administered daily with low-dose sildenafil treatment (5 mg/kg) immediately (within about 15 minutes) following each respective blast exposure and continuing daily to day seven, and rmbTBI subjects administered daily with high-dose sildenafil treatment (20 mg/kg) immediately (within about 15 minutes) following each respective blast exposure and continuing daily to day seven. Cortical capillaries were isolated at seven days post-rmbTBI and stained for zonula occludens-1 (ZO-1) and DAPI.

Cognitive Performance. The effect of sildenafil treatment on cognitive performance was assessed by comparing sham subjects administered daily with vehicle, rmbTBI subjects administered daily with vehicle immediately (within about 15 minutes) following each respective blast exposure and continuing to day three, rmbTBI subjects administered daily with low-dose sildenafil treatment (5 mg/kg) immediately (within about 15 minutes) following each respective blast exposure and continuing to day three, and rmbTBI subjects administered daily with high-dose sildenafil treatment (20 mg/kg) immediately (within about 15 minutes) following each respective blast exposure and continuing to day 3. Novel object recognition task was used to assay cognitive performance following rmbTBI. The first trial involved the exposure of the animal to identical “familiar” objects for five minutes. In the second trial, animals were exposed to a “familiar” object (same object used in the first task) and a “novel” object for five minutes. Trials took place in an opaque black acrylic box with dimensions 80×80×36 cm and animal behavior was tracked using Etho Vision XT™ tracking software. Precautions were taken to clean the chamber between the trials and have the experimenter leave the room during the experiment. For analysis, a recognition index was calculated for each trial (time spent exploring the familiar object relative to the novel object divided by total time exploring objects during each trial). A ratio of 0.5 indicated equal exploration of both objects during the trial. Results were assessed one week after blast exposure.

### Results

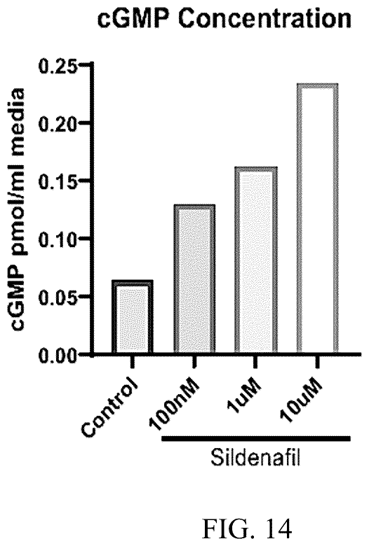

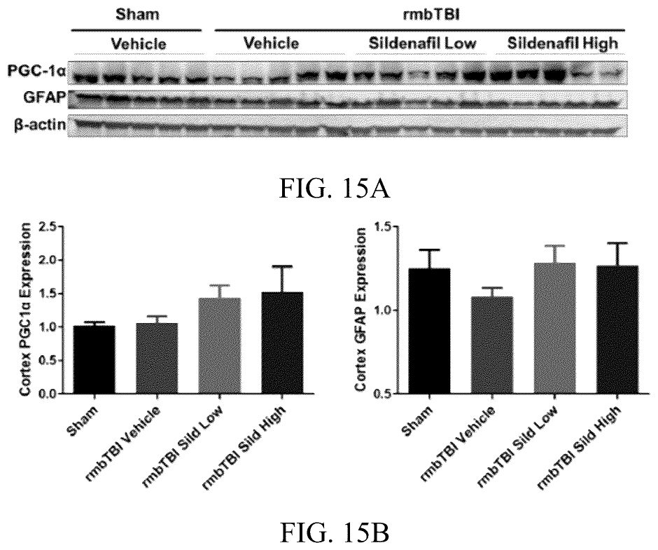

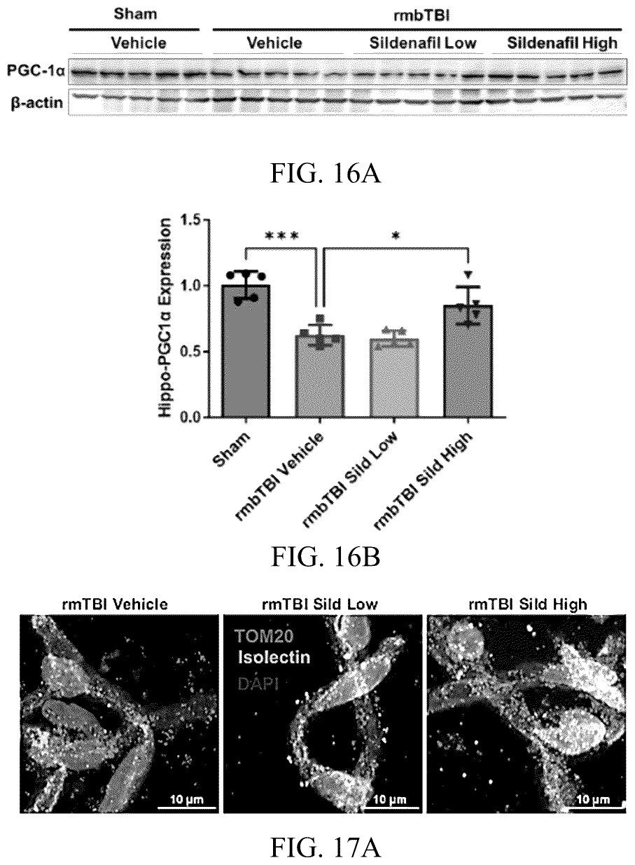

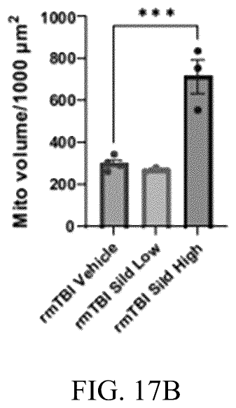

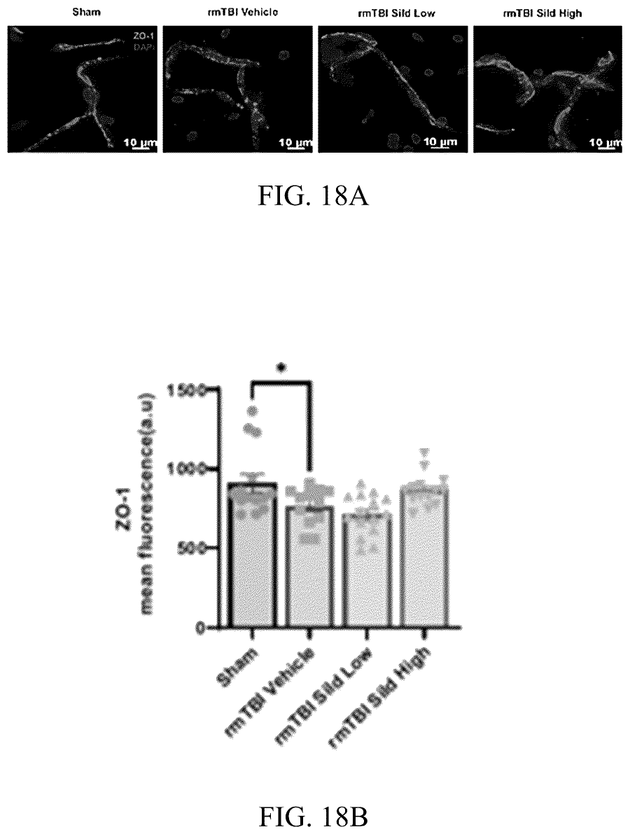

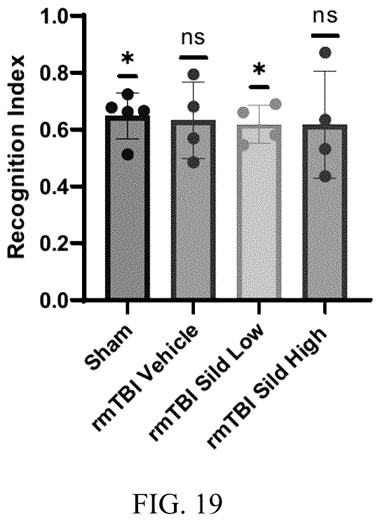

Sildenafil was found to induce increased cGMP in isolated capillaries (FIG. 14). Low-dose and high-dose sildenafil treatment increased both PGC-1α and GFAP expression in cortex tissue of rmbTBI subjects as compared to rmbTBI vehicle, thus indicating low-dose and high-dose sildenafil treatment increased mitochondrial biogenesis and astrocyte homeostasis in the cortical tissue of rmbTBI subjects (FIGS. 15A-15B). A significant decrease in hippocampal-PGC-1α expression was observed between sham and rmbTBI vehicle subjects (FIGS. 16A-16B). High-dose sildenafil treatment was, however, found to significantly increase PGC-1α expression as compared to rmbTBI vehicle subjects (FIGS. 16A-16B), thus indicating that higher dosages of sildenafil promote increases in mitochondrial biogenesis in the hippocampal tissue of rmbTBI subjects. As shown in FIGS. 17A and 17B, TOM20 expression was also found to be significantly increased in high-dose sildenafil treated rmbTBI subjects as compared to rmbTBI vehicle, thus indicating PDE5 inhibition facilitated by higher-dosages of sildenafil increase capillary-specific mitochondrial content in rmbTBI subjects. Tight junction protein ZO-1 was found to be significantly decreased rmbTBI vehicle subjects as compared to sham (FIGS. 18A-18B). High-dose treated rmbTBI subjects were found, however, to exhibit increased ZO-1 expression as compared to rmbTBI vehicle, thus indicating that higher dosages of sildenafil may serve to increase tight junction integrity (FIGS. 18A-18B). Finally, sham subjects and rmbTBI subjects treated with low-dose sildenafil were found to have a significantly increased recognition index as compared to chance, thus suggesting that sildenafil treatment can be effective in improving cognitive performance following rmbTBI (FIG. 19).

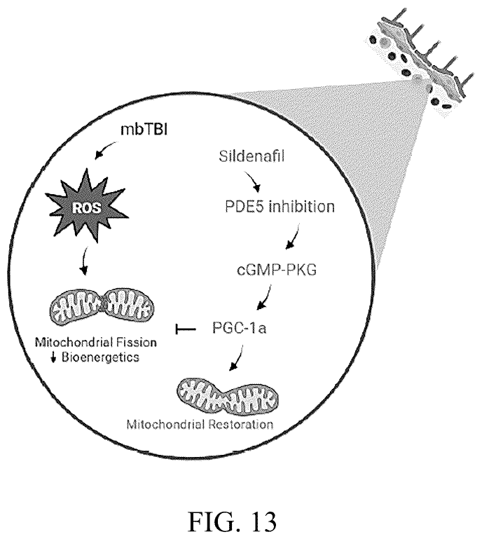

The increases in cGMP and PGC-1α observed in the studies underlying this example supports a mitochondrial-centric mechanism of sildenafil pharmacodynamics. Without wishing to be bound by theory, it is believed that increased cGMP and PGC-1α promotes mitochondrial bioenergetics. It is thus believed, that PDE5 inhibition as effectuated by sildenafil treatment, and the corresponding increases in cGMP and PGC-1α resulting therefrom, can facilitate mitochondrial biogenesis, thus overcoming oxidative stress and mitochondrial dysfunction caused by mild blast exposure (FIG. 13). The results of the studies underlying this example further suggest that in vivo sildenafil treatment can improve hippocampal levels of PGC-1α, which is a master regulator of mitochondrial biogenesis, mitochondrial content in isolated cortical capillaries, tight junction protein levels, and cognitive function.

### Example 4: Promotion of NAD/NADH Metabolism Following rmbTBI

### Methods and Materials

Animal Procedure. Male Sprague-Dawley rats (Charles River Laboratories, Wilmington, MA, USA), aged 8-10 weeks and weighing approximately 250-300 g, randomly assigned into experimental groups (n=5/group), including: (1) Sham group without blast treatment (vehicle via oral gavage daily for 7 days); (2), rmbTBI-Vehicle group (vehicle via oral gavage daily for 7 days, with administration occurring immediately following each respective blast exposure and continuing to day 7); and (3) rmbTBI-Sildenafil group (20 mg/kg sildenafil via oral gavage daily for 7 days, with administration occurring immediately following each respective blast exposure and continuing to day 7). The rmbTBI blast procedure was performed on day 0 and day 1 (24 h interval) (i.e., two blasts total) using a McMillan Blast Device at delivering 11 psi peak static overpressure blast wave. On day 8, the rats were euthanized without the use of isoflurane to preserve endogenous metabolites. The brain was rapidly removed, and the hippocampus and cortex were dissected, snap-frozen in liquid nitrogen, and stored at −80° C. until further analysis. Frozen brain tissues were submitted for liquid chromatography-mass spectrometry (LC-MS) analysis to quantify metabolites and assess biochemical alteration.

Sample Prep for LC-MS. All tissues were handled on liquid nitrogen when removed from cryostorage at −80° C. Brain samples were removed from cryostorage and pulverized to 10 μm particles in liquid N₂ using a Freezer/Mill Cryogenic Grinder (model 6875D, SPEX SamplePrep, Metuchen, NJ, USA). Approximately 25 mg of powdered tissue was removed with a micro spatula to a new tube and extracted with 50% MeOH (1% formic acid). Following addition of MeOH, mixtures were placed on ice for 20 min and briefly vortexed at 5 min intervals. Following centrifugation at 24,000×g for 10 min at 4° C. the aqueous phase containing polar metabolites was filtered to remove lipids using Agilent captiva EMR-Lipid filter under positive pressure manifold. The resulting filtrate was fully dried at 10-3 mBar using a SpeedVac (Thermo Fisher Scientific, Waltham, MA, USA) to evaporate remaining MeOH then reconstituted in 5% acetonitrile, normalized to mass of tissue used for extraction.

High-Performance Liquid Chromatograph (HPLC) Parameters. Metabolite separation was achieved using the Agilent 1290 Infinity II Stainless HPLC system equipped with an InfinityLab Poroshell 120 HILIC-Z column. The mobile phase consisted of solvent A (water with 10 mM ammonium acetate and 0.1% medronic acid) and solvent B (acetonitrile with 10 mM ammonium acetate). The flow rate set at 0.400 ml/min and gradient elution program were as set forth in TABLE 1.

TABLE 1
Gradient elution program for HPLC.

| Time | % B |
|---|---|
| 0 | 90 |
| 1.00 | 90 |
| 8.00 | 78 |
| 12.00 | 60 |
| 15.00 | 10 |
| 18.00 | 10 |
| 19.00 | 90 |
| 23.00 | 90 |

MS/MS Parameters. Analysis performed on Agilent 6495C LC/TQ with the following parameters: ion mode set at both positive and negative, gas temperature 200 C, drying gas flow 14 L/min, nebulizer gas at 50 psi, sheath gas temperature at 375 C, sheath gas flow at 12 L/min, capillary voltage set to (+) 3000/(−) 2500 V and nozzle voltage set at 0 V.

Data Analysis. After data acquisition, initial data analysis performed on Agilent MassHunter Quantitative Analysis for peak identification and quantification. Relative abundance of each metabolite was then exported into excel and statistical analysis performed using Metaboanalyst (https://www.metaboanalyst.ca). Graphs were made using GraphPad Prism 9.

### Results

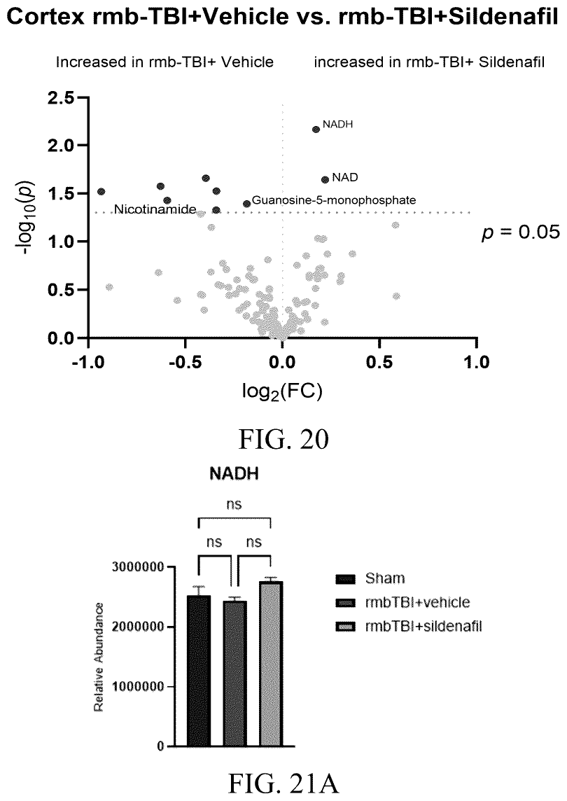

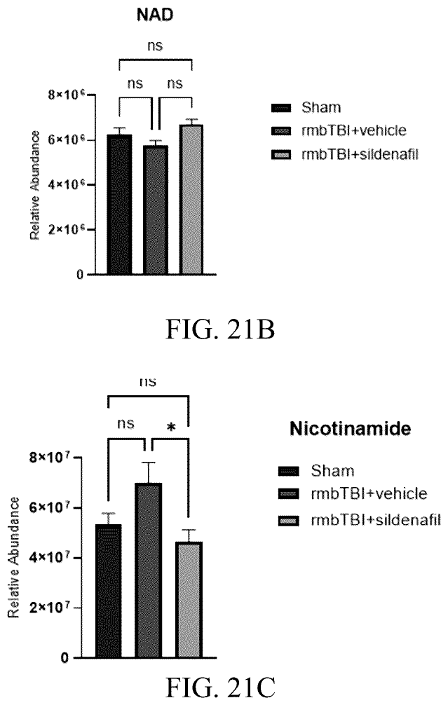

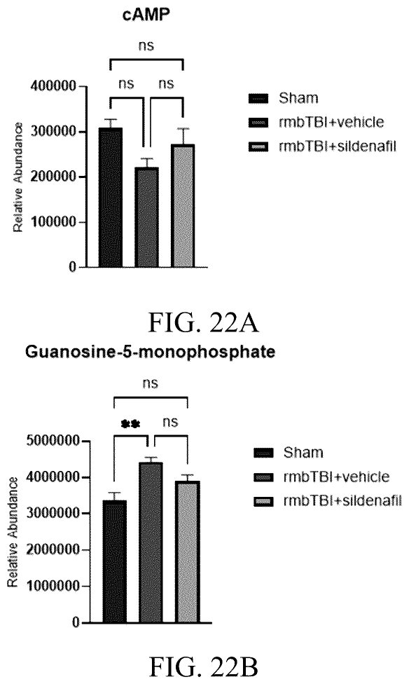

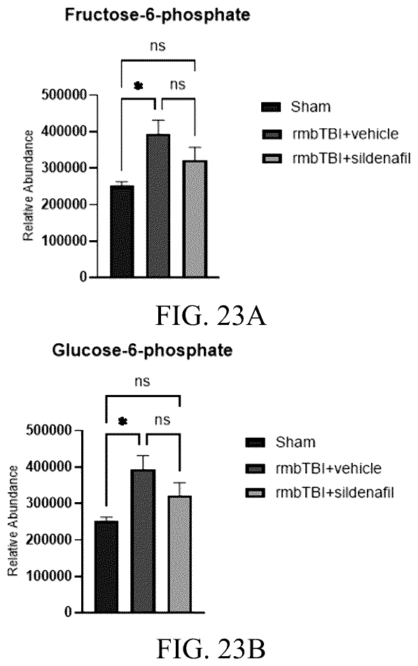

The cortex metabolic profile of rmbTBI subjects treated with vehicle versus those treated with sildenafil revealed key metabolic shifts associated with rmbTBI and the effects of sildenafil treatment. In this regard, decreases in nicotinamide adenine dinucleotide (NAD⁺), NADH, and cAMP were observed in rmbTBI vehicle subjects as compared to sham subjects (FIGS. 21A, 21B, and 22A). Sildenafil treatment was, however, found to increase NAD⁺, NADH, and cAMP relative to rmbTBI vehicle subjects (FIGS. 20, 21A, 21B, and 22A). An increase in nicotinamide, guanosine-5-monophasphate, fructose-6-phosphate, and glucose-6-phosphate were observed in rmbTBI vehicle subjects as compared to sham subjects (FIGS. 21C, 22B, 23A, and 23B). Sildenafil treatment was, however, found to decrease nicotinamide, guanosine-5-monophasphate, fructose-6-phosphate, and glucose-6-phosphate relative to rmbTBI vehicle subjects (FIGS. 20, 21C, 22B, 23A, and 23B).

These results indicate that key metabolites, such as nicotinamide and glucose-6-phosphate, can be restored through sildenafil treatment at seven days post injury. These results further suggest that sildenafil promotes NAD⁺/NADH metabolism by decreasing nicotinamide levels that are altered post injury. A reduction in nicotinamide levels, which drives increased NAD⁺/NADH metabolism, following sildenafil treatment can have several neurovascular benefits in the context of mbTBI or rmbTBI. NAD⁺ is essential for mitochondrial oxidative metabolism. Enhancing NAD⁺ availability promotes the activity of key enzymes like sirtuins (particularly SIRT1 and SIRT3), which regulate mitochondrial biogenesis and endothelial function. In the brain, especially in injured microvasculature, improved NAD⁺/NADH balance supports mitochondrial respiration in endothelial cells, stabilizing energy production and reducing the production of reactive oxygen species (ROS). This improvement in mitochondrial efficiency within brain capillaries may guard against oxidative stress—a known contributor to blood-brain barrier (BBB) breakdown and neuroinflammation following blast exposure.

Regarding post-traumatic-stress disorder (PTSD), mitochondrial dysfunction and neurovascular instability can contribute to persistent stress-related symptoms by disrupting the regulation of limbic system activity (e.g., in the amygdala and hippocampus). By improving endothelial mitochondrial function and reducing oxidative burden, sildenafil-induced enhancement of NAD⁺ metabolism may mitigate pathological metabolic and inflammatory cascades that sustain PTSD symptoms post-TBI.

### Example 5: Chronic Effect of rmbTBI and Sildenafil Treatment

### Methods and Materials

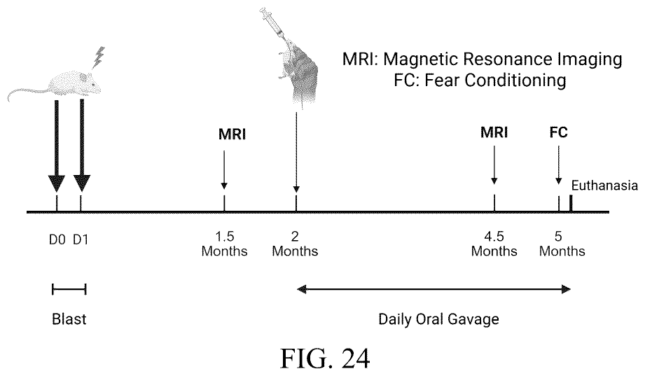

Animal Procedure. 8-12 week old male Sprague Dawley rats (purchased from Charles River) randomly assigned into experimental groups: 1) Sham group without blast treatment, total n=12 rats (n=6 received 20 mg/kg sildenafil and n=6 received vehicle via oral gavage daily from 2 months to 5 months post-blast injury); 2) rmbTBI-Vehicle group, total n=11 rats (vehicle via oral gavage daily from 2 months to 5 months post-blast injury); 3) rmbTBI-Sildenafil group, total n=12 rats (20 mg/kg sildenafil via oral gavage daily from 2 months to 5 months post-blast injury). MRI (magnetic resonance imaging) was performed at 1.5 and 4.5 months post-blast. Fear conditioning test was performed at 5 months post-blast. At the end of the experiment, rats were euthanized and the brain tissue was collected after transcardial perfusion with PBS (FIG. 24). One hemisphere of the brain was snap frozen with liquid nitrogen for brain capillary isolation while another hemisphere of the brain was fixed with 4% paraformaldehyde (PFA). After 48 hours of fixation, the brain tissue was placed into PBS containing 30% sucrose for 72 hours. The sucrose saturated brain tissue was flash frozen in-25 to −35° C. isopentane and stored at −20° C. or proceeded to sectioning.

Brain tissue Immunostaining. The PFA fixed and processed half rat brain tissue from all groups was sliced into 40-micron thickness. Using floating tissue staining method, the brain slices were incubated with ImmunoDNA Retriever buffer with Citrate (Ref. BSB 0020) at 95 degrees for 5 min and allowing cooling at RT for 20 min. After washing with PBS for 10 min twice, the brain slices were permeabilized with a blocking buffer (0.2% Triton X-100, 10% normal horse serum, and 1% BSA in PBST) for one hour and then incubated with primary antibodies: rabbit anti-ionized calcium-binding adaptor molecule 1 (IBA1) (1:250 dilution, FUJIFilm, #016-20002) and Monoclonal Anti-Glial Fibrillary Acidic Protein (GFAP) (1:250 dilution, Sigma, G3893) overnight at 4 degrees. The slices were then incubated with the secondary antibodies: Alexa Fluor 488 goat anti-mouse (1:500 dilution, Fisher Scientific, catalog no. A11001) and Alexa fluor 594 anti-rabbit (1:500 dilution, Fisher Scientific, catalog no. A-21207) at RT for one hour. The tissues were subsequently washed with PBST, mounted onto glass slides, and coverslipped with VECTASHIELD® HardSet™ Antifade Mounting Medium with DAPI.

Brain Tissue HALO Analysis. Slides were Axio scanned and loaded onto Indica Lab's Halo AI v4.0.5107.407 for analysis. Regions of interest (ROIs) were defined by annotations outlining the cortex, hippocampus, amygdala, and corpus callosum. A HALO AI MiniNet classifier was trained to recognize blood vessels with a resolution of 2.0 μm/px and a minimum object size of 350 um². A “blood vessel” class had first priority and an “other tissue” class had second priority, and the classifier was trained to reach a cross-entropy of approximately 0.07. GraphPad Prism 10.4.1 was used to generate graphs and run statistical analyses.

Brain Capillary Isolation and Immunostaining. Half frozen rat brain from all groups was homogenized with isolation buffer (pH7.4 DPBS containing 5.5 mM glucose, 1 mM sodium pyruvate, and 1% BSA). The homogenate was centrifuged at 1000×g for 10 min at 4° C. to remove the supernatant. The pellet was resuspended with lymphocyte separation medium (25-072-CV; Corning Life Sciences) and centrifuged at 4500×g for 20 min at 4° C. to remove myelin and some cells. The pellet sample was resuspended in isolation buffer, followed by filtering with a 70-micron μm membrane filter (15-1070; Tisch Scientific) to remove larger vessels and an 18-micron membrane filter (ME17233; Tisch Scientific) to trap the capillaries. The harvested capillaries were released from the 18-micron filter membrane with isolation buffer. After evaluating the quality and concentration of capillary suspension under a bright field microscope, which may contain small amount of large vessel and cells, the isolated capillary sample was ready for the immunostaining analysis. Around 100 μl of isolated capillary suspension was loaded into 8-well chamber slide and incubated at room temperature (RT) for 1-2 hours depending on the capillary concentration. The adhered capillary sample was fixed with 4% paraformaldehyde for 10 min at RT. After permeabilization and blocking with buffer (0.2% Triton X-100 in PBST, 1% BSA, and 10% normal horse serum) for 1 hour at RT, the capillary slides were incubated overnight at 4° C. with primary antibodies for zona occludens-1 (ZO-1) (tight junction marker, 1:250 dilution, Themo Fisher, #40-2200) and for monoclonal OXPHOS (mitochondria marker, 1:100 dilution, Abcam, ab110413). After wash, the capillary slides were incubated with secondary antibodies: Alexa Fluor 488 goat anti-mouse (1:500 dilution, Fisher Scientific, A11001) and Alexa Fluor 594 anti-rabbit (1:500 dilution, Fisher Scientific, A-21207) for 2 hours at RT. After washing, the capillary samples were mounted with a Vectashield Hard-Set Antifade Mounting Medium with DAPI (H-1500-10; Vector laboratories). Images were acquired using a Nikon confocal microscope.

The expression of ZO-1 was quantified by measuring the mean fluorescence intensity by tracing the capillary under NIS-Elements analysis software. The total capillary area was measured by using NIS-Elements. For mitochondrial volume, the OXPHOS fluorescence from Z-stack images was quantified using Imaris (X64 9.6.1) using surface creation tool. A smoothed surface with background subtraction (local contrast) was used to separate the mitochondria from the background. A split-touching object was enabled to separate individual adjacent mitochondria. The template file was used to bulk-process all the images from different groups. Mitochondrial volume (sum of total mitochondrial volume detected by Imaris surface detection tool) was normalized to the total capillary area and expressed in 1000 μm2. The correlation between ZO-1 expression and Mitochondrial-Volume was analyzed with XY analyses and graphed by using GraphPad Prism 10.4.1. p value smaller than 0.05 is significant.

Magnetic Resonance Imaging (MRI). MRI scanning was performed at 1.5 months and 4.5 months post-blast injury. Briefly, rats were placed under anesthesia with 4% isoflurane before placing it in the MRI scanner. Physiological monitoring was recorded every 10 min (with normal range 300-450 bpm) and rat body temperature was maintained while scanning under anesthesia. The experiments were performed in a Bruker 7T preclinical MRI scanner with a volume transmit coil (Bruker T12053V3) and 2×2 surface receiver coil (Bruker T11483V3). T2 weighted anatomical images were acquired with a spin echo sequence (TR/TE 2750/33 ms; in-plane resolution 98×98 μm2; slice thickness 1 mm; RARE factor 8). Single slice pCASL images were acquired with an echo planar image (TR/TE 4000/16 ms; 260×260 μm2; slice thickness 1 mm; 3000 ms labeling time; 300 ms transit time). Labeling efficiency was calculated from a pCASL-encoded fast low-angle shot (FLASH) sequence (TR/TE=250/3.5 ms; in plane resolution 182×182 μm2; slice thickness 1.5 mm; flip angle 80° labeling time 200 ms; transit time 0 ms) image of the carotid artery just below the split and 5 mm above the labeling slice. The MRI data analysis of cerebral blood flow (CBF) was done by using FSL software. Data were graphed with GraphPad Prism 10.4.1.

Fear Conditioning Test. Contextual Fear Conditioning paradigm was performed over 4 days. On the acquisition day, rats were placed in a foot shock chamber with a camera within a sound-attenuating chamber and were allowed to explore freely for 2 min. After 2 min, a total of 6 electric shocks (0.75 mA, 2 s duration) were delivered at intervals of 60 s through the testing chamber floor, with shock delivery controlled by Med associates Inc. VideoFreeze™ Video Fear Conditioning Software. The rats then remained in the chamber for an additional 2 min with no shocks delivered before being removed. On test days 2-4, rats were placed for 8 min in same chambers as on acquisition day, with no shocks delivered, to determine the extent of contextual fear learning and extinction of contextual fear conditioning. Video footage was later scored for freezing behavior using Med associates Inc. VideoFreeze™ Video Fear Conditioning Software. Freezing behavior was defined as complete immobility except for minor movements required for respiration. The freezing episodes on four days were graphed with GraphPad Prism 10.4.1.

### Results

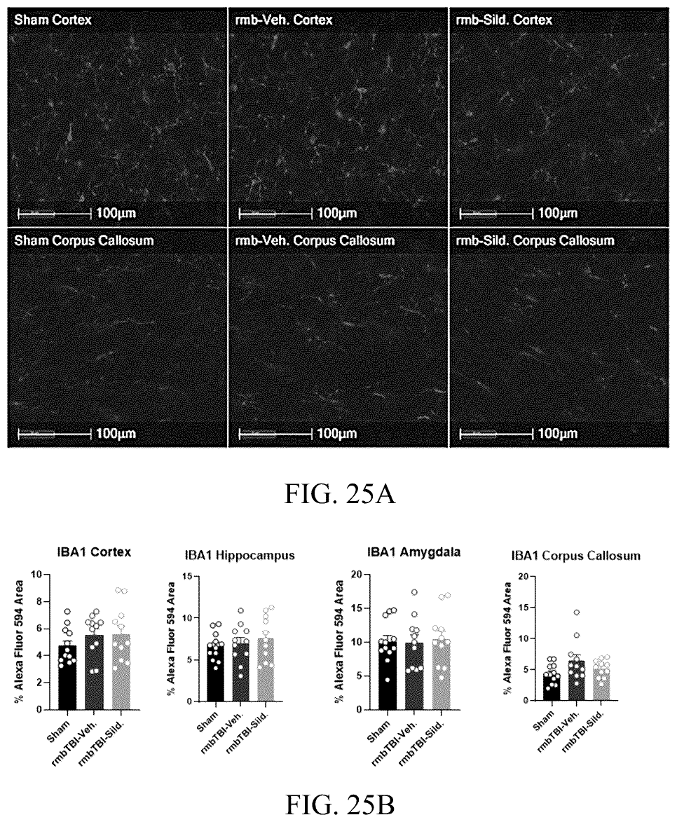

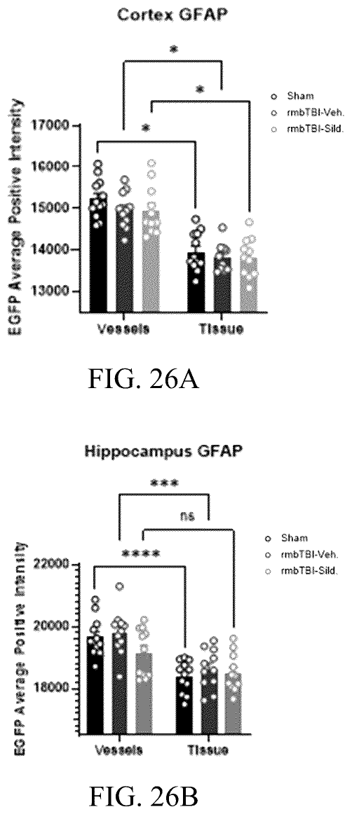

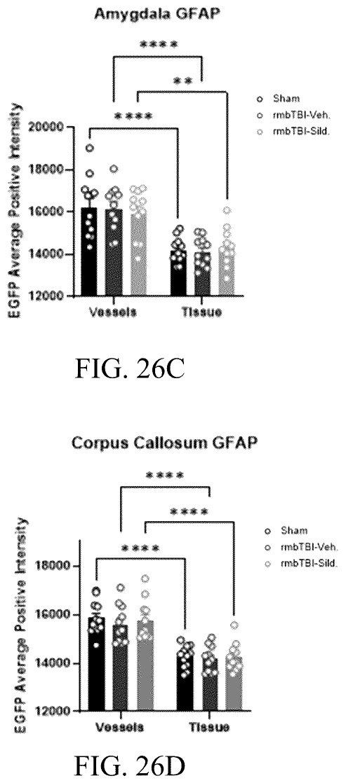

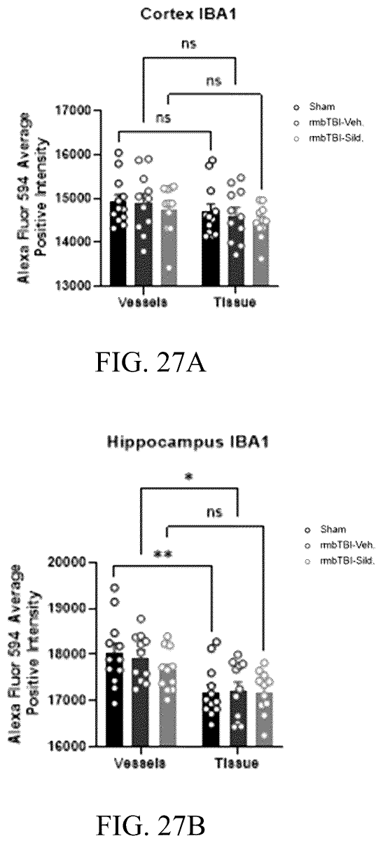

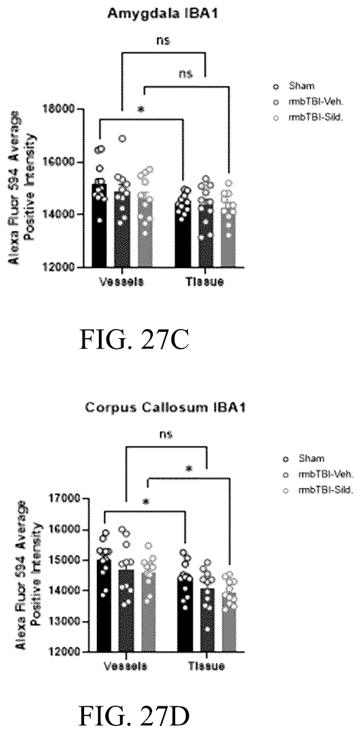

The effect of sildenafil with respect to IBA1 expression was inconclusive as no significant differences in the cortex, hippocampus, amygdala, and corpus callosum between sham and rmbTBI vehicle groups was observed at two months post-blast exposure (FIGS. 25A-25B). GFAP expression was increased in the blood vessels in each test group compared to other tissues in the cortex, hippocampus, amygdala, and corpus callosum (FIGS. 26A-26D). Likewise, IBA1 expression was increased in the blood vessels in each test group compared to other tissues in the cortex, hippocampus, amygdala, and corpus callosum (FIGS. 27A-27D).

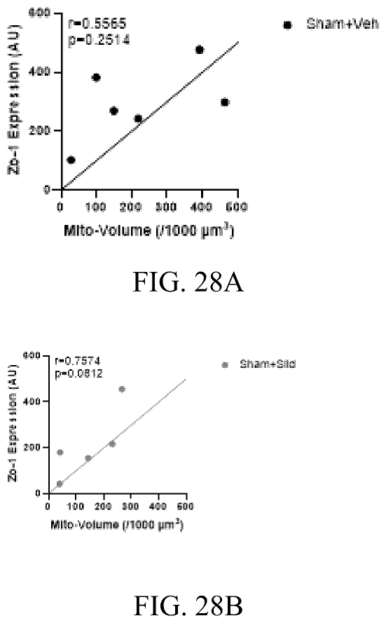

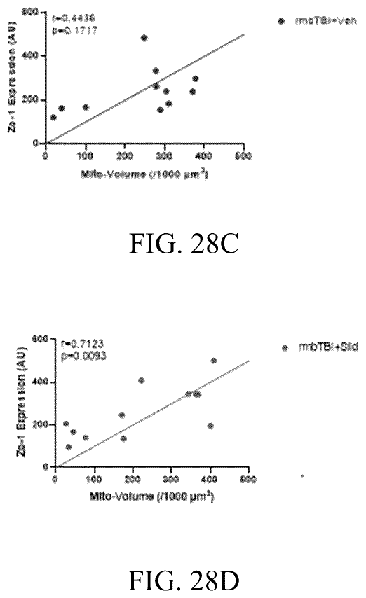

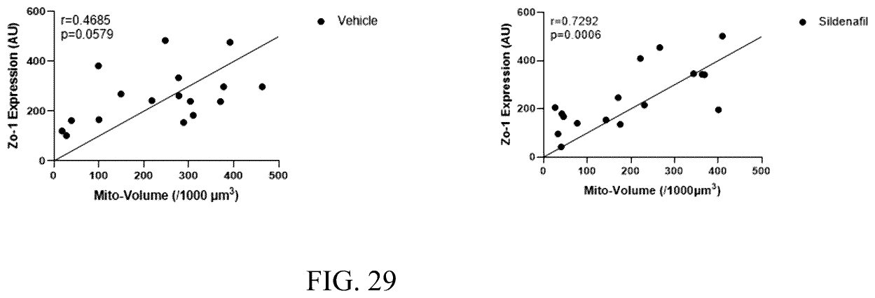

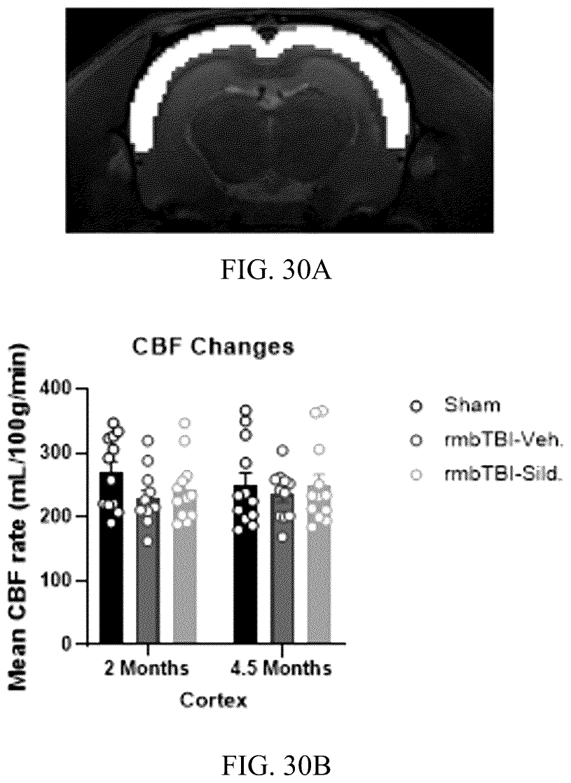

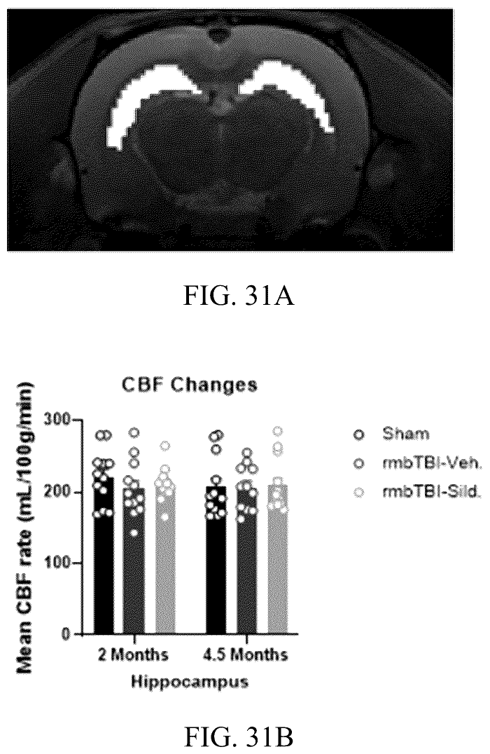

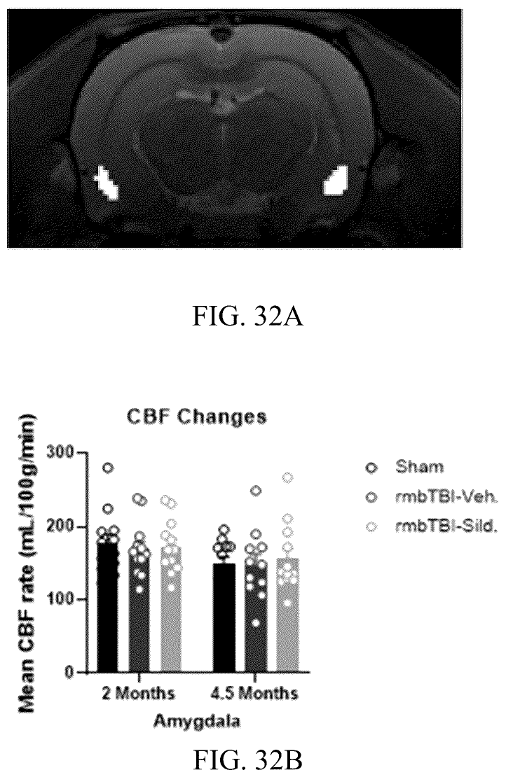

Sildenafil treatment strengthened the correlation between chronic tight junction integrity and mitochondrial content in brain capillaries following rmbTBI. To determine the long-term relationship between mitochondrial content and tight junction (TJ) integrity in the cerebrovascular endothelium, we quantified these parameters in brain capillaries at 5 months post-repetitive mild blast traumatic brain injury (rmbTBI). Capillary mitochondrial content was assessed by analysis of OXPHOS immunoreactivity, while tight junction integrity was measured via ZO-1 expression. In the vehicle-treated rmbTBI group, no significant correlation was observed between mitochondrial content and tight junction protein expression (FIGS. 28A, 28C, 29), suggesting persistent dissociation between energy metabolism and barrier structure in the chronic post-injury state. In contrast, animals treated chronically with sildenafil exhibited a significant positive correlation between capillary mitochondrial content and tight junction integrity (FIGS. 28B, 28D, 29). This finding suggests that sildenafil restores or reinforces a functional coupling between endothelial mitochondrial support and barrier protein expression. Notably, this correlation was not present in vehicle-treated rmbTBI, indicating that sildenafil facilitates a unique neurovascular repair mechanism that persists into the chronic phase post-injury. Together, these findings demonstrate that long-term sildenafil treatment enhances the association between metabolic capacity and barrier stability in the injured cerebrovasculature, potentially contributing to improved vascular health and neuroprotection following rmbTBI.

Cerebral blood flow remained unchanged following rmbTBI with or without sildenafil treatment. Quantitative MRI arterial spin labeling (ASL) was used to assess cerebral blood flow (CBF) in the cortex, hippocampus, and amygdala at 2 and 4.5 months post-rmbTBI. No significant differences in regional CBF were observed between sham, rmbTBI+vehicle, or rmbTBI+sildenafil groups at either time point (p>0.05 for all regions and comparisons). These findings indicate that rmbTBI, with or without chronic sildenafil treatment, does not alter steady-state CBF in key brain regions commonly implicated in neurovascular and behavioral dysfunction.

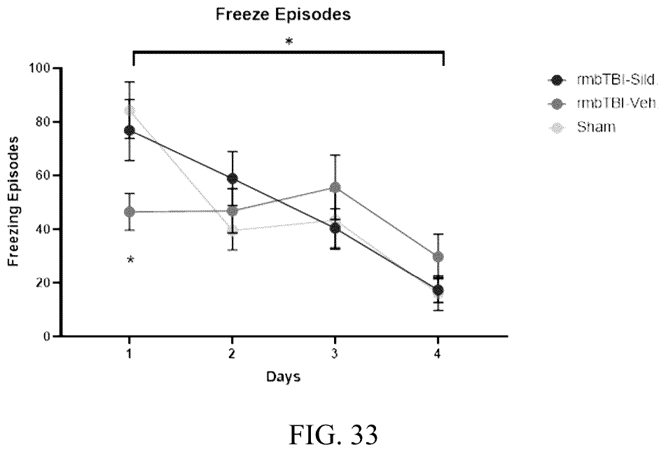

In the fear conditioning analysis, the sham group was statistically elevated at day one compared to rmbTBI vehicle, and sham and rmbTBI sildenafil groups showed a significant time-related decrease of freezing episodes, consistent with extinction, that were not observed in rmbTBI vehicle groups (FIG. 33). This finding is indicative of behavioral dysregulation after rmbTBI that is normalized by sildenafil treatment.

Overall, rmbTBI did not result in robust behavioral, imaging, or pathological deficits by five months post-injury. However, a link between mitochondrial content and tight junction repair was observed, further demonstrating that sildenafil could act through mitochondrial mechanisms to promote vascular recovery after rmbTBI.

The strengthened correlation between mitochondrial volume and tight junction proteins following sildenafil treatment suggests an interdependence between mitochondrial health and BBB structural integrity. Tight junction proteins are critical for maintaining BBB function, and their expression and organization can be disrupted by oxidative stress and metabolic dysfunction. Sildenafil's ability to enhance this correlation indicates improved vascular integrity for vessels that have increased mitochondrial content. Since the assembly and maintenance of tight junctions are energy-dependent processes, better mitochondrial function supports tight junction complexes. Functionally, this mitochondrial-tight junction coupling enhances the BBB. This is particularly important after mbTBI or rmbTBI, where chronic vascular dysfunction can lead to neuroinflammation, synaptic dysfunction, and neuropsychiatric symptoms, including PTSD. Thus, by reinforcing the metabolic support for barrier integrity, sildenafil may contribute to vascular normalization and a reduction in PTSD-related symptomatology in injured subjects. This finding is supported by behavioral improvement in the fear extinction paradigm.

Given that vascular and mitochondrial disruptions emerge early following low-level blast exposure and the lack of chronic deficits after low-level blast, our findings suggest that administering sildenafil during the acute window (as in Examples 3 and 4) is more advantageous. Early mitochondrial and vascular deficits following blast exposure are likely to initiate maladaptive neuronal circuit remodeling that becomes increasingly difficult to reverse in the chronic phase. Disruption of brain capillary mitochondrial function and blood-brain barrier integrity can lead to sustained oxidative stress, neuroinflammation, and impaired neurovascular coupling. These early pathophysiological events can drive aberrant synaptic pruning, altered connectivity, and the persistence of behavioral and cognitive deficits. By intervening early with PDE5 inhibition facilitated by sildenafil or another PDE5 inhibitor-when these vascular and metabolic impairments first emerge-downstream circuit-level changes that underlie chronic neuropsychiatric symptoms may be prevented or mitigated. As such, it is believed there is a meaningful therapeutic advantage realized by early post-blast-injury treatment.

### REFERENCES

All publications, patents, and patent applications mentioned in this specification are herein incorporated by reference to the same extent as if each individual publication, patent, or patent application was specifically and individually indicated to be incorporated by reference, including the references set forth in the following list:
1. Hubbard, W. B., Greenberg, S., Norris, C., et al. (2017). Distinguishing the unique neuropathological profile of blast polytrauma. Oxid Med Cell Longev 2017, 5175249.
2. Meyer, E. C., La Bash, H., DeBeer, B. B., et al. (2018). Psychological inflexibility predicts ptsd symptom severity in war veterans after accounting for established ptsd risk factors and personality. Psychol Trauma.
3. Kintzle, S., Barr, N., Corletto, G., et al. (2018). Ptsd in U.S. Veterans: The role of social connectedness, combat experience and discharge. Healthcare (Basel) 6.
4. Wold, B. L., Nolan, R., Nwafor, D., et al. (2018). Post-traumatic stress disorder delineating the progression and underlying mechanisms following blast traumatic brain injury. J Neurosci Neuropharmacol 4.
5. Baker, M. T., Moring, J. C., Hale, W. J., et al. (2018). Acute assessment of traumatic brain injury and post-traumatic stress after exposure to a deployment-related explosive blast. Mil Med.
6. Armed Forces Health Surveillance, C. (2013). Anxiety disorders, active component, U.S. Armed forces, 2000-2012. MSMR 20, 2-6; discussion 4-6.
7. Walker, W. C., Franke, L. M., McDonald, S. D., et al. (2015). Prevalence of mental health conditions after military blast exposure, their co-occurrence, and their relation to mild traumatic brain injury. Brain Inj 29, 15811588.
8. Carr, W., Polejaeva, E., Grome, A., et al. (2015). Relation of repeated low-level blast exposure with symptomology similar to concussion. J Head Trauma Rehabil 30, 47-55.
9. Elder, G. A., Stone, J. R. and Ahlers, S. T. (2014). Effects of low-level blast exposure on the nervous system: Is there really a controversy? Frontiers in Neurology 5, 269.
10. Perez-Garcia, G., Gama Sosa, M. A., De Gasperi, R., et al. (2018). Chronic post-traumatic stress disorder-related traits in a rat model of low-level blast exposure. Behavioural Brain Research 340, 117-125.
11. Gil, S., Caspi, Y., Ben-Ari, I. Z., et al. (2005). Does memory of a traumatic event increase the risk for posttraumatic stress disorder in patients with traumatic brain injury? A prospective study. Am J Psychiatry 162, 963-969.
12. Harvey, A. G., Brewin, C. R., Jones, C., et al. (2003). Coexistence of posttraumatic stress disorder and traumatic brain injury: Towards a resolution of the paradox. J Int Neuropsychol Soc 9, 663-676.
13. Sajja, V. S., Hubbard, W. B. and VandeVord, P. J. (2015). Subacute oxidative stress and glial reactivity in the amygdala are associated with increased anxiety following blast neurotrauma. Shock 44 Suppl 1, 71-78.
14. Ivanov, I., Fernandez, C., Mitsis, E. M., et al. (2017). Blast exposure, white matter integrity, and cognitive function in iraq and afghanistan combat veterans. Frontiers in Neurology 8, 127.
15. Carr, W., Stone, J. R., Walilko, T., et al. (2016). Repeated low-level blast exposure: A descriptive human subjects study. Military Medicine 181, 28-39.
16. Reneer, D. V., Hisel, R. D., Hoffman, J. M., et al. (2011). A multi-mode shock tube for investigation of blast-induced traumatic brain injury. J Neurotrauma 28, 95-104.
17. Bonnette, S., Diekfuss, J. A., Kiefer, A. W., et al. (2018). A jugular vein compression collar prevents alterations of endogenous electrocortical dynamics following blast exposure during special weapons and tactical (swat) breacher training. Exp Brain Res.
18. Kamimori, G. H., LaValle, C. R., Eonta, S. E., et al. (2018). Longitudinal investigation of neurotrauma serum biomarkers, behavioral characterization, and brain imaging in soldiers following repeated low-level blast exposure (new zealand breacher study). Mil Med 183, 28-33.
19. Trudeau, D. L., Anderson, J., Hansen, L. M., et al. (1998). Findings of mild traumatic brain injury in combat veterans with ptsd and a history of blast concussion. J Neuropsychiatry Clin Neurosci 10, 308-313.
20. Elder, G. A., Dorr, N. P., De Gasperi, R., et al. (2012). Blast exposure induces post-traumatic stress disorder-related traits in a rat model of mild traumatic brain injury. J Neurotrauma 29, 2564-2575.
21. Perez-Garcia, G., Gama Sosa, M. A., De Gasperi, R., et al. (2016). Exposure to a predator scent induces chronic behavioral changes in rats previously exposed to low-level blast: Implications for the relationship of blast-related tbi to ptsd. Frontiers in Neurology 7, 176.
22. Gama Sosa, M. A., De Gasperi, R., Janssen, P. L., et al. (2014). Selective vulnerability of the cerebral vasculature to blast injury in a rat model of mild traumatic brain injury. Acta Neuropathol Commun 2, 67.
23. Logsdon, A. F., Meabon, J. S., Cline, M. M., et al. (2018). Blast exposure elicits blood-brain barrier disruption and repair mediated by tight junction integrity and nitric oxide dependent processes. Sci Rep 8, 11344.
24. Shetty, A. K., Mishra, V., Kodali, M., et al. (2014). Blood brain barrier dysfunction and delayed neurological deficits in mild traumatic brain injury induced by blast shock waves. Front Cell Neurosci 8, 232.
25. Perez-Polo, J. R., Rea, H. C., Johnson, K. M., et al. (2015). A rodent model of mild traumatic brain blast injury. J Neurosci Res 93, 549-561.
26. Elder, G. A., Gama Sosa, M. A., De Gasperi, R., et al. (2015). Vascular and inflammatory factors in the pathophysiology of blast-induced brain injury. Front Neurol 6, 48.
27. Yeoh, S., Bell, E. D. and Monson, K. L. (2013). Distribution of blood-brain barrier disruption in primary blast injury. Ann Biomed Eng 41, 2206-2214.
28. Gama Sosa, M. A., De Gasperi, R., Paulino, A. J., et al. (2013). Blast overpressure induces shear-related injuries in the brain of rats exposed to a mild traumatic brain injury. Acta Neuropathol Commun 1, 51.
29. Meabon, J. S., Huber, B. R., Cross, D. J., et al. (2016). Repetitive blast exposure in mice and combat veterans causes persistent cerebellar dysfunction. Sci Transl Med 8, 321ra326.
30. Readnower, R. D., Chavko, M., Adeeb, S., et al. (2010). Increase in blood-brain barrier permeability, oxidative stress, and activated microglia in a rat model of blast-induced traumatic brain injury. J Neurosci Res 88, 3530-3539.
31. Abdul-Muneer, P. M., Schuetz, H., Wang, F., et al. (2013). Induction of oxidative and nitrosative damage leads to cerebrovascular inflammation in an animal model of mild traumatic brain injury induced by primary blast. Free Radic Biol Med 60, 282-291.
32. Hue, C. D., Cho, F. S., Cao, S., et al. (2016). Time course and size of blood-brain barrier opening in a mouse model of blast-induced traumatic brain injury. J Neurotrauma 33, 1202-1211.
33. Rajkowska, G., Hughes, J., Stockmeier, C. A., et al. (2013). Coverage of blood vessels by astrocytic endfeet is reduced in major depressive disorder. Biol Psychiatry 73, 613-621.
34. Gama Sosa, M. A., De Gasperi, R., Perez Garcia, G. S., et al. (2019). Low-level blast exposure disrupts gliovascular and neurovascular connections and induces a chronic vascular pathology in rat brain. Acta Neuropathol Commun 7, 6.
35. Hubbard, W. B., Lashof-Sullivan, M., Greenberg, S., et al. (2018). Hemostatic nanoparticles increase survival, mitigate neuropathology and alleviate anxiety in a rodent blast trauma model. Sci Rep 8, 10622.
36. Mahan, A. L. and Ressler, K. J. (2012). Fear conditioning, synaptic plasticity and the amygdala: Implications for posttraumatic stress disorder. Trends Neurosci 35, 24-35.
37. Liberzon, I. and Sripada, C. S. (2008). The functional neuroanatomy of ptsd: A critical review. Prog Brain Res 167, 151-169.
38. Gama Sosa, M. A., De Gasperi, R., Perez Garcia, G. S., et al. (2017). Lack of chronic neuroinflammation in the absence of focal hemorrhage in a rat model of low-energy blast-induced tbi. Acta Neuropathol Commun 5, 80.
39. Neigh, G. N., Owens, M. J., Taylor, W. R., et al. (2010). Changes in the vascular area fraction of the hippocampus and amygdala are induced by prenatal dexamethasone and/or adult stress. J Cereb Blood Flow Metab 30, 1100-1104.
40. Ratliff, W. A., Mervis, R. F., Citron, B. A., et al. (2019). Mild blast-related tbi in a mouse model alters amygdalar neurostructure and circuitry. Exp Neurol 315, 9-14.
41. Carr, W., Yarnell, A. M., Ong, R., et al. (2015). Ubiquitin carboxy-terminal hydrolase-11 as a serum neurotrauma biomarker for exposure to occupational low-level blast. Frontiers in Neurology 6, 49.
42. Karnati, H. K., Garcia, J. H., Tweedie, D., et al. (2018). Neuronal enriched extracellular vesicle proteins as biomarkers for brain traumatic injury. Journal of Neurotrauma.
43. Di Pietro, V., Yakoub, K. M., Scarpa, U., et al. (2018). Microrna signature of traumatic brain injury: From the biomarker discovery to the point-of-care. Front Neurol 9, 429.
44. Ramirez, S. H., Andrews, A. M., Paul, D., et al. (2018). Extracellular vesicles: Mediators and biomarkers of pathology along cns barriers. Fluids Barriers CNS 15, 19.
45. Raghavan, V., Bhomia, M., Torres, I., et al. (2017). Hypothesis: Exosomal micrornas as potential biomarkers for schizophrenia. Med Hypotheses 103, 21-25.
46. Balakathiresan, N. S., Chandran, R., Bhomia, M., et al. (2014). Serum and amygdala microrna signatures of posttraumatic stress: Fear correlation and biomarker potential. J Psychiatr Res 57, 65-73.
47. Wang, W. X., Visavadiya, N. P., Pandya, J. D., et al. (2015). Mitochondria-associated micrornas in rat hippocampus following traumatic brain injury. Exp Neurol 265, 84-93.
48. Guillotte, A. R., Herbert, J. P., Madsen, R., et al. (2018). Effects of platelet dysfunction and platelet transfusion on outcomes in traumatic brain injury patients. Brain Inj 32, 1849-1857.
49. Martin, G. E., Xia, B., Kim, Y., et al. (2018). Platelet function changes in a time-dependent manner following traumatic brain injury in a murine model. Shock 50, 551-556.
50. Epstein, E. L., Martindale, S. L., Va Mid-Atlantic Mirecc, W., et al. (2019). Posttraumatic stress disorder and traumatic brain injury: Sex differences in veterans. Psychiatry Res 274, 105-111.
51. Mac Donald, C. L., Johnson, A. M., Wierzechowski, L., et al. (2014). Prospectively assessed clinical outcomes in concussive blast vs nonblast traumatic brain injury among evacuated us military personnel. JAMA Neurol 71, 994-1002.
52. Ahlers, S. T., Vasserman-Stokes, E., Shaughness, M. C., et al. (2012). Assessment of the effects of acute and repeated exposure to blast overpressure in rodents: Toward a greater understanding of blast and the potential ramifications for injury in humans exposed to blast. Front Neurol 3, 32.
53. Hubbard, W. B., Bailey, Z., Vande Vord P. J. (2016). Cellular mechanisms and behavioral outcomes in blast-induced neurotrauma: Comparing experimental set-ups. Methods in Molecular Biology 1462, 119-138.
54. Sajja, V. S., Hubbard, W. B., Hall, C. S., et al. (2015). Enduring deficits in memory and neuronal pathology after blast-induced traumatic brain injury. Sci Rep 5, 15075.
55. Bevins, R. A. and Besheer, J. (2006). Object recognition in rats and mice: A one-trial non-matching-to-sample learning task to study ‘recognition memory’. Nat Protoc 1, 1306-1311.
56. Darwish, H., Mahmood, A., Schallert, T., et al. (2014). Simvastatin and environmental enrichment effect on recognition and temporal order memory after mild-to-moderate traumatic brain injury. Brain Inj 28, 211-226.
57. Pelz, J., Hartig, W., Weise, C., et al. (2013). Endothelial barrier antigen-immunoreactivity is conversely associated with blood-brain barrier dysfunction after embolic stroke in rats. Eur J Histochem 57, e38.
58. Westin, J. E., Lindgren, H. S., Gardi, J., et al. (2006). Endothelial proliferation and increased blood-brain barrier permeability in the basal ganglia in a rat model of 3,4-dihydroxyphenyl-1-alanine-induced dyskinesia. J Neurosci 26, 9448-9461.
59. Li, Y., Song, Y., Zhao, L., et al. (2008). Direct labeling and visualization of blood vessels with lipophilic carbocyanine dye dii. Nature protocols 3, 1703-1708.
60. Hubbard, W. B., Joseph, B., Spry, M., et al. (2018). Acute mitochondrial impairment underlies prolonged cellular dysfunction after repeated mild traumatic brain injuries. J Neurotrauma.
61. Robinson, S., Berglass, J. B., Denson, J. L., et al. (2017). Microstructural and microglial changes after repetitive mild traumatic brain injury in mice. J Neurosci Res 95, 1025-1035.
62. Glushakova, O. Y., Johnson, D. and Hayes, R. L. (2014). Delayed increases in microvascular pathology after experimental traumatic brain injury are associated with prolonged inflammation, blood-brain barrier disruption, and progressive white matter damage. J Neurotrauma 31, 1180-1193.
63. Goldstein, L. E., Rasmusson, A. M., Bunney, B. S., et al. (1996). Role of the amygdala in the coordination of behavioral, neuroendocrine, and prefrontal cortical monoamine responses to psychological stress in the rat. J Neurosci 16, 4787-4798.
64. Johansen, J. P., Cain, C. K., Ostroff, L. E., et al. (2011). Molecular mechanisms of fear learning and memory. Cell 147, 509-524.
65. Matthews, S. C., Spadoni, A. D., Lohr, J. B., et al. (2012). Diffusion tensor imaging evidence of white matter disruption associated with loss versus alteration of consciousness in warfighters exposed to combat in operations enduring and iraqi freedom. Psychiatry Res 204, 149-154.
66. Knutson, K. M., Rakowsky, S. T., Solomon, J., et al. (2013). Injured brain regions associated with anxiety in vietnam veterans. Neuropsychologia 51, 686-694.
67. Huang, M. X., Yurgil, K. A., Robb, A., et al. (2014). Voxel-wise resting-state meg source magnitude imaging study reveals neurocircuitry abnormality in active-duty service members and veterans with ptsd. Neuroimage Clin 5, 408-419.
68. Davis, J. J., Walter, K. H., Chard, K. M., et al. (2013). Treatment adherence in cognitive processing therapy for combat-related ptsd with history of mild tbi. Rehabil Psychol 58, 36-42.
69. Morey, R. A., Gold, A. L., LaBar, K. S., et al. (2012). Amygdala volume changes in posttraumatic stress disorder in a large case-controlled veterans group. Arch Gen Psychiatry 69, 1169-1178.
70. Wahlsten, D. (2001). Standardizing tests of mouse behavior: Reasons, recommendations, and reality. Physiol Behav 73, 695-704.
71. Christmas, A. J. and Maxwell, D. R. (1970). A comparison of the effects of some benzodiazepines and other drugs on aggressive and exploratory behaviour in mice and rats. Neuropharmacology 9, 17-29.
72. Crawley, J. and Goodwin, F. K. (1980). Preliminary report of a simple animal behavior model for the anxiolytic effects of benzodiazepines. Pharmacol Biochem Behav 13, 167-170.
73. File, S. E. and Hyde, J. R. (1978). Can social interaction be used to measure anxiety? Br J Pharmacol 62, 19-24.
74. Smith, D. H., Hicks, R. R., Johnson, V. E., et al. (2015). Pre-clinical traumatic brain injury common data elements: Toward a common language across laboratories. J Neurotrauma 32, 1725-1735.
75. Semple, B. D., Canchola, S. A. and Noble-Haeusslein, L. J. (2012). Deficits in social behavior emerge during development after pediatric traumatic brain injury in mice. J Neurotrauma 29, 2672-2683.
76. Dinkins, M. B., Enasko, J., Hernandez, C., et al. (2016). Neutral sphingomyelinase-2 deficiency ameliorates alzheimer's disease pathology and improves cognition in the 5×fad mouse. J Neurosci 36, 8653-8667.
77. Genovese, R. F., Simmons, L. P., Ahlers, S. T., et al. (2013). Effects of mild tbi from repeated blast overpressure on the expression and extinction of conditioned fear in rats. Neuroscience 254, 120-129.
78. Benton, R. L., Maddie, M. A., Minnillo, D. R., et al. (2008). Griffonia simplicifolia isolectin b4 identifies a specific subpopulation of angiogenic blood vessels following contusive spinal cord injury in the adult mouse. J Comp Neurol 507, 1031-1052.
79. Kenney, K., Qu, B. X., Lai, C., et al. (2018). Higher exosomal phosphorylated tau and total tau among veterans with combat-related repetitive chronic mild traumatic brain injury. Brain Inj 32, 1276-1284.
80. Song, H., Chen, M., Chen, C., et al. (2019). Proteomic analysis and biochemical correlates of mitochondrial dysfunction after low-intensity primary blast exposure. J Neurotrauma.
81. Vanderploeg, R. D., Belanger, H. G., Horner, R. D., et al. (2012). Health outcomes associated with military deployment: Mild traumatic brain injury, blast, trauma, and combat associations in the florida national guard. Arch Phys Med Rehabil 93, 1887-1895.
82. Cheng, J. S., Craft, R., Yu, G. Q., et al. (2014). Tau reduction diminishes spatial learning and memory deficits after mild repetitive traumatic brain injury in mice. PLOS One 9, e115765.
83. Aungst, S. L., Kabadi, S. V., Thompson, S. M., et al. (2014). Repeated mild traumatic brain injury causes chronic neuroinflammation, changes in hippocampal synaptic plasticity, and associated cognitive deficits. J Cereb Blood Flow Metab 34, 1223-1232.
84. Cao, R., Zhang, C., Mitkin, V. V., et al. (2019). Comprehensive characterization of cerebrovascular dysfunction in blast traumatic brain injury using photoacoustic microscopy. J Neurotrauma.
85. Lyons, D. N., Vekaria, H., Macheda, T., et al. (2018). A mild traumatic brain injury in mice produces lasting deficits in brain metabolism. J Neurotrauma 35, 2435-2447.
86. Fierstra, J., Sobczyk, O., Battisti-Charbonney, A., et al. (2013). Measuring cerebrovascular reactivity: What stimulus to use? J Physiol 591, 5809-5821.
87. Peskind, E. R., Petrie, E. C., Cross, D. J., et al. (2011). Cerebrocerebellar hypometabolism associated with repetitive blast exposure mild traumatic brain injury in 12 iraq war veterans with persistent post-concussive symptoms. Neuroimage 54 Suppl 1, S76-82.
88. Hubbard, W. B., Harwood, C. L., Geisler, J. G., et al. (2018). Mitochondrial uncoupling prodrug improves tissue sparing, cognitive outcome, and mitochondrial bioenergetics after traumatic brain injury in male mice. J Neurosci Res 96, 1677-1688.
89. Perez-Garcia, G., De Gasperi, R., Gama Sosa, M. A., et al. (2018). Ptsd-related behavioral traits in a rat model of blast-induced mtbi are reversed by the mglur2/3 receptor antagonist bci-838. eNeuro 5.
90. Mahmood, G., Mei, Z., Hojjat, H., et al. (2014). Therapeutic effect of sildenafil on blast-induced tinnitus and auditory impairment. Neuroscience 269, 367-382.
91. Kenney, K., Amyot, F., Moore, C., et al. (2018). Phosphodiesterase-5 inhibition potentiates cerebrovascular reactivity in chronic traumatic brain injury. Ann Clin Transl Neurol 5, 418-428.
92. Diaz-Arrastia, R., Kochanek, P. M., Bergold, P., et al. (2014). Pharmacotherapy of traumatic brain injury: State of the science and the road forward: Report of the department of defense neurotrauma pharmacology workgroup. J Neurotrauma 31, 135-158.
93. Venkat, P., Chopp, M., Zacharek, A., et al. (2018). Sildenafil treatment of vascular dementia in aged rats. Neurochem Int.
94. Zhang, R., Wang, Y., Zhang, L., et al. (2002). Sildenafil (viagra) induces neurogenesis and promotes functional recovery after stroke in rats. Stroke 33, 2675-2680.
95. Assis-Nascimento, P., Tsenkina, Y. and Liebl, D. J. (2018). Ephb3 signaling induces cortical endothelial cell death and disrupts the blood-brain barrier after traumatic brain injury. Cell Death Dis 9, 7.
96. Guilfoyle, M. R., Carpenter, K. L., Helmy, A., et al. (2015). Matrix metalloproteinase expression in contusional traumatic brain injury: A paired microdialysis study. J Neurotrauma 32, 1553-1559.
97. Dinkins, M. B., Dasgupta, S., Wang, G., et al. (2014). Exosome reduction in vivo is associated with lower amyloid plaque load in the 5×fad mouse model of alzheimer's disease. Neurobiol Aging 35, 1792-1800.
98. Taylor, D. D., Zacharias, W. and Gercel-Taylor, C. (2011). Exosome isolation for proteomic analyses and rna profiling. Methods Mol Biol 728, 235-246.
99. Tomer, R., Ye, L., Hsueh, B., et al. (2014). Advanced clarity for rapid and high-resolution imaging of intact tissues. Nature protocols 9, 1682-1697.
100. Saatman, K. E., et al., Classification of traumatic brain injury for targeted therapies. J Neurotrauma, 2008. 25 (7): p. 719-38.
101. Ponto, L. L., et al., Preliminary Investigation of Cerebral Blood Flow and Amyloid Burden in Veterans With and Without Combat-Related Traumatic Brain Injury. J Neuropsychiatry Clin Neurosci, 2016. 28 (2): p. 89-96.
102. Kenney, K., et al., Cerebral Vascular Injury in Traumatic Brain Injury. Exp Neurol, 2016. 275 Pt 3: p. 353-366.
103. Jullienne, A., et al., Chronic cerebrovascular dysfunction after traumatic brain injury. Journal of neuroscience research, 2016. 94 (7): p. 609-622.
104. Tagge, C. A., et al., Concussion, microvascular injury, and early tauopathy in young athletes after impact head injury and an impact concussion mouse model. Brain, 2018. 141 (2): p. 422-458.
105. Glushakova, O. Y., D. Johnson, and R. L. Hayes, Delayed Increases in Microvascular Pathology after Experimental Traumatic Brain Injury Are Associated with Prolonged Inflammation, Blood-Brain Barrier Disruption, and Progressive White Matter Damage. Journal of Neurotrauma, 2014. 31 (13): p. 1180-1193.
106. Hubbard, W. B., et al., Distinguishing the Unique Neuropathological Profile of Blast Polytrauma. Oxidative Medicine and Cellular Longevity, 2017. 2017: p. 5175249.
107. Johnson, V. E., et al., Mechanical disruption of the blood-brain barrier following experimental concussion. Acta Neuropathologica, 2018. 135 (5): p. 711-726.
108. Sahyouni, R., et al., Effects of concussion on the blood-brain barrier in humans and rodents. J Concussion, 2017. 1.
109. Yeoh, S., E. D. Bell, and K. L. Monson, Distribution of blood-brain barrier disruption in primary blast injury. Ann Biomed Eng, 2013. 41 (10): p. 2206-14.
110. Langen, U. H., S. Ayloo, and C. Gu, Development and Cell Biology of the Blood-Brain Barrier. Annu Rev Cell Dev Biol, 2019. 35: p. 591-613.
111. Keaney, J. and M. Campbell, The dynamic blood-brain barrier. FEBS J, 2015. 282 (21): p. 4067-79.
112. Brown, L. S., et al., Pericytes and Neurovascular Function in the Healthy and Diseased Brain. Front Cell Neurosci, 2019. 13: p. 282.
113. Posada-Duque, R. A., G. E. Barreto, and G. P. Cardona-Gomez, Protection after stroke: cellular effectors of neurovascular unit integrity. Front Cell Neurosci, 2014. 8: p. 231.
114. Abbott, N.J., Blood-brain barrier structure and function and the challenges for CNS drug delivery. J Inherit Metab Dis, 2013. 36 (3): p. 437-49.
115. Muldoon, L. L., et al., Immunologic privilege in the central nervous system and the blood-brain barrier. J Cereb Blood Flow Metab, 2013. 33 (1): p. 13-21.
116. Zhao, Z., et al., Cardiolipin-mediated procoagulant activity of mitochondria contributes to traumatic brain injury-associated coagulopathy in mice. Blood, 2016. 127 (22): p. 2763-72.
117. Shi, K., et al., Dissemination of brain inflammation in traumatic brain injury. Cell Mol Immunol, 2019. 16 (6): p. 523-530.
118. Nag, S., A. Kapadia, and D. J. Stewart, Review: molecular pathogenesis of blood-brain barrier breakdown in acute brain injury. Neuropathol Appl Neurobiol, 2011. 37 (1): p. 3-23.
119. Chodobski, A., B. J. Zink, and J. Szmydynger-Chodobska, Blood-brain barrier pathophysiology in traumatic brain injury. Translational stroke research, 2011. 2 (4): p. 492-516.
120. Shetty, A. K., et al., Blood brain barrier dysfunction and delayed neurological deficits in mild traumatic brain injury induced by blast shock waves. Front Cell Neurosci, 2014. 8: p. 232.
121. Agoston, D. V., Modeling the Long-Term Consequences of Repeated Blast-Induced Mild Traumatic Brain Injuries. Journal of Neurotrauma, 2017. 34 (S1): p. S-44-S-52.
122. Elder, G. A., et al., Vascular and inflammatory factors in the pathophysiology of blast-induced brain injury. Frontiers in neurology, 2015. 6: p. 48-48.
123. Gama Sosa, M. A., et al., Low-level blast exposure disrupts gliovascular and neurovascular connections and induces a chronic vascular pathology in rat brain. Acta Neuropathologica Communications, 2019. 7 (1): p. 6.
124. Salehi, A., J. H. Zhang, and A. Obenaus, Response of the cerebral vasculature following traumatic brain injury. Journal of cerebral blood flow and metabolism: official journal of the International Society of Cerebral Blood Flow and Metabolism, 2017. 37 (7): p. 2320-2339.
125. Prins, M. L., et al., Repeated mild traumatic brain injury: mechanisms of cerebral vulnerability. Journal of neurotrauma, 2013. 30 (1): p. 30-38.
126. Moeini, M., et al., Compromised microvascular oxygen delivery increases brain tissue vulnerability with age. Scientific Reports, 2018. 8 (1): p. 8219.
127. Sure, V. N., et al., A novel high-throughput assay for respiration in isolated brain capillaries reveals impaired mitochondrial function in the aged mice. Geroscience, 2018. 40 (4): p. 365-375.
128. Hu, H., et al., Mitochondrial Impairment in Cerebrovascular Endothelial Cells is Involved in the Correlation between Body Temperature and Stroke Severity. Aging and disease, 2016. 7 (1): p. 14-27.
129. Prakash, R. and S. T. Carmichael, Blood-brain barrier breakdown and neovascularization processes after stroke and traumatic brain injury. Current opinion in neurology, 2015. 28 (6): p. 556-564.
130. Amyot, F., et al., Assessment of cerebrovascular dysfunction after traumatic brain injury with fMRI and fNIRS. NeuroImage. Clinical, 2020. 25: p. 102086-102086.
131. Rana, P., et al., Oxidative stress contributes to cerebral metabolomic profile changes in animal model of blast-induced traumatic brain injury. Metabolomics, 2020. 16 (3): p. 39.
132. Readnower, R. D., et al., Increase in blood-brain barrier permeability, oxidative stress, and activated microglia in a rat model of blast-induced traumatic brain injury. J Neurosci Res, 2010. 88 (16): p. 3530-9.
133. Sajja, V. S. S. S., W. B. Hubbard, and P. J. Vande Vord, Subacute Oxidative Stress and Glial Reactivity in the Amygdala are Associated with Increased Anxiety Following Blast Neurotrauma. Shock, 2015. 44.
134. Abdul-Muneer, P. M., et al., Induction of oxidative and nitrosative damage leads to cerebrovascular inflammation in an animal model of mild traumatic brain injury induced by primary blast. Free Radic Biol Med, 2013. 60: p. 282-91.
135. Kuriakose, M., et al., Synergistic Role of Oxidative Stress and Blood-Brain Barrier Permeability as Injury Mechanisms in the Acute Pathophysiology of Blast-induced Neurotrauma. Scientific Reports, 2019. 9 (1): p. 7717.
136. Keller, J. N. and et al., 4-Hydroxynonenal, an aldehydic product of membrane lipid peroxidation, impairs glutamate transport and mitochondrial function in synaptosomes. Neuroscience., 1997. 80 (3): p. 685-96.
137. Keller, J. N., et al., Impairment of glucose and glutamate transport and induction of mitochondrial oxidative stress and dysfunction in synaptosomes by amyloid beta-peptide: role of the lipid peroxidation product 4-hydroxynonenal. J Neurochem, 1997. 69 (1): p. 273-84.
138. Mark, R. J., et al., A role for 4-hydroxynonenal, an aldehydic product of lipid peroxidation, in disruption of ion homeostasis and neuronal death induced by amyloid beta-peptide. J Neurochem, 1997. 68 (1): p. 255-64.
139. Sullivan, P. G., et al., Traumatic brain injury alters synaptic homeostasis: implications for impaired mitochondrial and transport function. J Neurotrauma, 1998. 15 (10): p. 789-98.
140. Logsdon, A. F., et al., Blast exposure elicits blood-brain barrier disruption and repair mediated by tight junction integrity and nitric oxide dependent processes. Sci Rep, 2018. 8 (1): p. 11344.
141. Kenney, K., et al., Phosphodiesterase-5 inhibition potentiates cerebrovascular reactivity in chronic traumatic brain injury. Ann Clin Transl Neurol, 2018. 5 (4): p. 418-428.
142. Diaz-Arrastia, R., et al., Pharmacotherapy of traumatic brain injury: state of the science and the road forward: report of the Department of Defense Neurotrauma Pharmacology Workgroup. J Neurotrauma, 2014. 31 (2): p. 135-58. 143. Rempe, R. G., et al., Matrix Metalloproteinase-Mediated Blood-Brain Barrier Dysfunction in Epilepsy. J Neurosci, 2018. 38 (18): p. 4301-4315.
144. Cao, R., et al., Comprehensive Characterization of Cerebrovascular Dysfunction in Blast Traumatic Brain Injury Using Photoacoustic Microscopy. J Neurotrauma, 2019.
145. Venkat, P., et al., Sildenafil treatment of vascular dementia in aged rats. Neurochem Int, 2018.
146. Li, X., X. Sun, and P. Carmeliet, Hallmarks of Endothelial Cell Metabolism in Health and Disease. Cell Metab, 2019. 30 (3): p. 414-433.
147. Reneer, D. V., et al., A multi-mode shock tube for investigation of blast-induced traumatic brain injury. J Neurotrauma, 2011. 28 (1): p. 95-104.
148. Carr, W., et al., Repeated Low-Level Blast Exposure: A Descriptive Human Subjects Study. Military Medicine, 2016. 181 (suppl_5): p. 28-39.
149. Hartz, A. M. S., et al., Isolation of Cerebral Capillaries from Fresh Human Brain Tissue. J Vis Exp, 2018 (139).
150. Hubbard, W. B., et al., Acute Mitochondrial Impairment Underlies Prolonged Cellular Dysfunction after Repeated Mild Traumatic Brain Injuries. J Neurotrauma, 2019. 36 (8): p. 1252-1263.
151. Vekaria, H. J., et al., Formoterol, a β2-adrenoreceptor agonist, induces mitochondrial biogenesis and promotes cognitive recovery after traumatic brain injury. Neurobiology of Disease, 2020. 140: p. 104866.
152. Carlson, Shaun W., et al., “Central Infusion of Insulin-Like Growth Factor-1 Increases Hippocampal Neurogenesis and Improves Neurobehavioral Function after Traumatic Brain Injury” J. Neurotrauma 2018 Jul. 1; 35 (13): 1467-1480.
153. Hubbard W B, Velmurugan G V, Brown E P, Sullivan P G. Resilience of females to acute blood-brain barrier damage and anxiety behavior following mild blast traumatic brain injury. Acta Neuropathologica Communications. 2022; 10 (1): 93. doi: 10.1186/s40478-022-01395-8.
154. Velmurugan G, Prajapati S, Tran S, Miller C, Burke B, Hubbard B. “Sex-Dependent Blood-Brain Barrier Alterations Following Repeated Mild Blast Traumatic Brain Injury at Varying Inter-Injury Intervals.” in Experimental Neurology. 2025 May 30:115325. doi: 10.1016/j.expneurol.2025.115325.
155. Velmurugan, GV, Vekaria, H. J., Hartz A M S, Bauer B, Hubbard, WB. Oxidative stress alters mitochondrial homeostasis in isolated brain capillaries. Fluids Barriers CNS 21, 81 (2024). https://doi.org/10.1186/s12987-024-00579-9.

It will be understood that various details of the presently disclosed subject matter can be changed without departing from the scope of the subject matter disclosed herein. Furthermore, the foregoing description is for the purpose of illustration only, and not for the purpose of limitation.

What is claimed is:

1. A method for treating low-level blast traumatic brain injury, comprising:
administering a phosphodiesterase-5 (PDE5) inhibitor to a subject following exposure of the subject to one or more low-level blasts having a force less than 17 pounds per square inch.

2. The method of claim 1, wherein the PDE5 inhibitor is sildenafil.

3. The method of claim 1, wherein the PDE5 inhibitor is administered to the subject within 24 hours following exposure of the subject to a low-level blast of the one or more low-level blasts.

4. The method of claim 1, wherein the PDE5 inhibitor is administered to the subject within one hour following exposure of the subject to a low-level blast of the one or more low-level blasts.

5. The method of claim 1, wherein administering the PDE5 inhibitor comprises multiple administrations of the PDE5 inhibitor to the subject.

6. The method of claim 5, wherein administering the PDE5 inhibitor comprises administering the PDE5 inhibitor to the subject daily for a period of at least seven days.

7. The method of claim 1, wherein the one or more low-level blasts comprises multiple low-level blasts.

8. The method of claim 7, wherein administration of the PDE5 inhibitor is first administered to the subject following exposure to a last-occurring low-level blast of the multiple low-level blasts.

9. The method of claim 7, wherein a first administration of the PDE5 inhibitor is administered to the subject following exposure of the subject to a first low-level blast, and a second administration of the PDE5 inhibitor is administered to the subject following exposure of the subject to a second low-level blast occurring after the first administration of the PDE5 inhibitor to the subject.

10. The method of claim 1, wherein the subject is without chronic traumatic brain injury prior to exposure of the one or more low-level blasts.

11. A method for mitigating the effects of blast injury on the vascular integrity of a subject, comprising:
administering a phosphodiesterase-5 (PDE5) inhibitor to the subject following exposure of the subject to one or more low-level blasts to thereby increase at least one of brain capillary respiration, mitochondrial respiration, mitochondrial density, mitochondrial biogenesis, astrocyte level or homeostasis, and tight junction protein expression in a brain of a subject.

12. The method of claim 11, wherein the PDE5 inhibitor is sildenafil.

13. The method of claim 11, wherein the PDE5 inhibitor is administered to the subject within 24 hours following exposure of the subject to a low-level blast of the one or more low-level blasts.

14. The method of claim 11, wherein administration of the PDE5 inhibitor increases at least one of cyclic guanosine monophosphate (cGMP), proliferator-activated receptor gamma coactivator 1-alpha (PGC-1α), glial fibrillary acidic protein (GFAP), translocase of the outer mitochondrial membrane complex subunit 20 (TOM20), and zonula occludens-1 (ZO-1) in the subject.

15. The method of claim 11, wherein the one or more blasts comprises multiple low-level blasts, and wherein administration of the PDE5 inhibitor is first administered to the subject following exposure to a last-occurring low-level blast of the multiple low-level blasts.

16. The method of claim 11, wherein the one or more blasts comprises multiple low-level blasts, and wherein a first administration of the PDE5 inhibitor is administered to the subject following exposure of the subject to a first low-level blast, and a second administration of the PDE5 inhibitor is administered to the subject following exposure of the subject to a second low-level blast occurring after the first administration of the PDE5 inhibitor to the subject.

17. The method of claim 11, wherein administering the PDE5 inhibitor comprises administering the PDE5 inhibitor daily to the subject for a period of at least seven days.

18. A method for inducing a metabolic change in a subject, comprising:
administering a phosphodiesterase-5 (PDE5) inhibitor to a subject following exposure of the subject to one or more low-level blasts to thereby increase NAD⁺/NADH metabolism in the subject.

19. The method of claim 18, wherein administration of the PDE5 inhibitor reduces nicotinamide in the subject.

20. The method of claim 18, wherein the PDE5 inhibitor is sildenafil.
